(19) United States
(12) Patent Application Publication
(10) Pub. No.: US 2015/0353909 A1
(43) Pub. Date: Dec. 10, 2015

(54) ISOLATED POLYPEPTIDES, KITS COMPRISING THE SAME AND USES THEREOF

(71) Applicant: ACADEMIA SINICA, Taipei (TW)

(72) Inventors: Hong-Yong FU, Taipei (TW); Ramalingam RADJACOMMARE, Taipei (TW)

(21) Appl. No.: 14/729,737

(22) Filed: Jun. 3, 2015

Related U.S. Application Data
(60) Provisional application No. 62/009,957, filed on Jun. 10, 2014.

(51) Int. Cl. C12N 9/48; C12P 21/06

(52) U.S. Cl. CPC C12N 9/485; C12P 21/06; C12Y 304/00

(57) ABSTRACT

Disclosed herein are isolated polypeptides, kits comprising the same, and uses thereof. The polypeptides are respectively isolated from Arabidopsis thaliana and are capable of catalyzing the cleavage of a peptide linkage located between the C-terminus of the distal ubiquitin (UB) and any of the first methionine (M1), the 48^{th} (K48) or the 63^{rd} lysine (K63) of the proximal UB in a UB chain, which is conjugated to a substrate protein.

### BACKGROUND OF THE INVENTION

1. Field of the Invention

The present disclosure in general relates to the field of peptidase activity, more particularly to the cleavage of a peptide linkage between ubiquitin and a substrate protein, i.e., deubiquitination.

2. Description of Related Art

The function of a protein is regulated via various means in a cell; one way is via the conjugation and de-conjugation of a modifier protein ubiquitin (UB) to a target protein, e.g., ubiqutination or deubiquitination. The covalent modification of proteins by UB plays a central role in diverse cellular pathways such as cell cycle progression, signal transduction, transcriptional regulation, stress response, endocytosis, apoptosis and etc. Ubiquitination of a protein is catalyzed by a cascade of three enzymes. First, UB is activated by UB-activating enzyme (E1), which forms a thiol-ester bond with UB C-terminus. Then, UB is transferred to UB-conjugating enzyme (E2). Finally, UB C-terminus is ligated to the lysine residue of the target protein with the aid of UB ligases (E3). Target protein can be mono-ubiquitinated or multi-ubiquitinated by successive conjugation of UB C-terminus to the prior one through eight different linkages, which include M1, K6, K11, K27, K29, K33, K48 and K63 linkages.

While ubiqutination is important in diverse cellular pathways, the reverse reaction (i.e., deubiquitination), which is catalyzed by a diverse set of deubiquitination enzymes (DUBs), is equally important for modulating the ubiquitinated protein's activity and fate. The DUBs are proteases that specifically cleave the peptide bond between UBs or between UB C-terminus and covalently attached polypeptides. DUBs have been grouped into 5 classes, and 4 of these classes are cysteine proteases, including UB C-terminal hydrolases (UCHs), UB-specific processing (UBP) proteases, ovarian tumor (OTU)-related proteases and Josephin/Machado-Joseph disease (MJD) proteases. UBPs possess catalytic triad residues in highly conserved cysteine box and histidine box. UCHs have similar catalytic triad residues in two conserved cysteine and histidine boxes. As to OTUs, they also have a catalytic triad comparable to the other families in cysteine and histidine boxes.

In the model plant, Arabidopsis thaliana (A. thaliana), the function and biochemical properties of OTU DUBs have not yet been characterized. Inventors of this application unexpectedly identify 12 A. thaliana OTU loci and have analyzed the encoded proteins in vitro to determine their preferences for various types of UB chain linkages. Results of this study confirmed that 7 out of 12 proteins encoded from the identified A. thaliana OTU loci are active DUBs, and thus are useful in methods and/or kits involving deubiquitination of a substrate protein.

### SUMMARY

The present disclosure is based, at least in part, unexpected discovery of 7 A. thaliana OTU proteins respectively possess DUB activity, hence are useful in methods and/or kits involving deubiquitination of a substrate protein.

Accordingly, it is the first aspect of this disclosure to provide an isolated A. thaliana polypeptide or a homologue thereof capable of cleaving a peptide linkage between the C-terminus of the distal UB and any of the first methionine (M1), the 48^{th} (K48) or the 63^{rd} lysine (K63) of the proximal UB in a UB chain conjugated with a protein. The isolated polypeptide comprises an amino acid sequence at least 85% identical to any of SEQ ID NO: 1 (OTU1, Gene Bank Access NO: JQ013442), SEQ ID NO: 2 (OTU2, Gene Bank Access NO: JQ013443), SEQ ID NO: 3 (OTU3, Gene Bank Access NO: JQ013444), SEQ ID NO: 4 (OTU4c, Gene Bank Access NO: JQ013446), SEQ ID NO: 5 (OTU4c-NΔ, Gene Bank Access NO: JQ013446), SEQ ID NO: 6 (OTU7a, Gene Bank Access NO: JQ013452), SEQ ID NO: 7 (OTU7b, Gene Bank Access NO: JQ013453), SEQ ID NO: 8 (OTU9, Gene Bank Access NO: JQ013455), or SEQ ID NO: 9 (OTU10, Gene Bank Access NO: JQ013456).

In one example, the isolated polypeptide has the amino acid sequence at least 85% identical to SEQ ID NO: 1, and mutations at amino acid residues 92, 89 and 288 of the isolated polypeptide abolish its activity to cleave the peptide linkage located between the C-terminus of the distal UB and the M1, K48, or K63 of the proximal UB in the UB chain.

In another example, the isolated polypeptide has the amino acid sequence at least 85% identical to SEQ ID NO: 4, and a mutation at amino acid residue 136 of the isolated polypeptide abolish its activity to cleave the peptide linkage located between the C-terminus of the distal UB and the K48 or K63 of the proximal UB in the UB chain. In one preferred example, the isolated polypeptide has the amino acid sequence identical to SEQ ID NO: 5.

In still another example, the isolated polypeptide has the amino acid sequence at least 85% identical to SEQ ID NO: 6, and a mutation at amino acid residue 48 of the isolated polypeptide abolish its activity to cleave the peptide linkage located between the C-terminus of the distal UB and the K48 or K63 of the proximal UB in the UB chain.

In a further example, the isolated polypeptide has the amino acid sequence at least 85% identical to SEQ ID NO: 8 or 9, and cleaves the peptide linkage located between the C-terminus of the distal UB and the K63 of the proximal UB in the UB chain at about pH 9 to 9.5.

According to some embodiments of the present disclosure, the isolated polypeptide has the amino acid sequence at least 85% identical to SEQ ID NO: 1 (OTU1), SEQ ID NO: 3 (OTU3), SEQ ID NO: 4 (OTU4c), SEQ ID NO: 6 (OTU7a), SEQ ID NO: 8 (OTU9), or SEQ ID NO: 9 (OTU10); and is capable of cleaving the peptide linkage located between the C-terminus of the distal UB and the K48 or 63 of the proximal UB in the UB chain at about pH 7 to 8. In other embodiments, the isolated polypeptide has the amino acid sequence at least 85% identical to SEQ ID NO: 2, and two mutations at amino acid residues 63 and 99, or 179 and 195 of the isolated polypeptide abolish its activity to bind ubiquitin.

It is the second aspect of this disclosure to provide a kit for cleaving a peptide linkage located between the C-terminus of the distal UB and any of the first methionine (M1), the 48^{th} (K48) or the 63^{rd} lysine (K63) of the proximal UB in a UB chain, which is conjugated with a substrate protein. The kit includes a container; at least one isolated polypeptide of the present disclosure; and a legend associated with the container and indicating how to use the isolated polypeptide to direct the cleavage of the afore-mentioned peptide linkage.

According to embodiments of the present disclosure, the substrate protein may be any protein modified with ubiquitin and/or its fragment; and the peptide linkage is located between the C-terminus of the distal ubiquitin (UB) and any of the first methionine (M1), the 48^{th} (K48) or the 63^{rd} lysine (K63) of the proximal UB in the UB chain; and the isolated polypeptide has an amino acid sequence at least 85% identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

It is therefore the third aspect of this disclosure to provide a method of deubiquitinating a substrate protein. The method includes the step of subjecting the substrate protein having a conjugated UB chain to the action of an isolated polypeptide of the present disclosure for a sufficient period of time, so as to allow the cleavage of the peptide linkage between the C-terminus of the distal ubiquitin (UB) and any of the first methionine (M1), the 48^{th} (K48) or the 63^{rd} lysine (K63) of the proximal UB in the UB chain, wherein the isolated polypeptide has an amino acid sequence at least 85% identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 is a schematic diagram illustrating the phylogenetic tree of the OTU-proteins from A. thaliana, O. sativa, S. cerevisiae, and H. sapiens in accordance with one embodiment of the present disclosure;

FIG. 2 illustrates the A. thaliana OTU-containing proteins display distinctive preference for K48- and K63-linked UB tetramers in accordance with one embodiment of the present disclosure;

FIG. 3 illustrates the relative cleavage activities of A. thaliana OTU1, OTU3, and OTU4c against linear (tetra) and K48- or K63-linked UB tetramers in accordance with one embodiment of the present disclosure;

FIG. 4 illustrates the relative cleavage activities of A. thaliana OTU3, OTU4c, OTU7a, OTU9, and OTU10 for UB- and RUB-fused GST in accordance with one embodiment of the present disclosure;

FIG. 5 illustrates the inhibition of A. thaliana OTU1 by cysteine protease inhibitors in accordance with one embodiment of the present disclosure;

FIGS. 6A-6F illustrate the requirement of the conserved catalytic triad for the deubiquitylation activities of A. thaliana OTU1, OTU4c and OTU7a in accordance with one embodiment of the present disclosure;

FIGS. 7A-7B illustrate the association of A. thaliana OTU2 and OTLD1 with isopeptide bond-linked UB chains and their preference for different linkage types in accordance with one embodiment of the present disclosure;

FIG. 8A is a schematic diagram illustrating the site-specific and deletion variants of OTU2 that were used to delineate the domains and residues involved in UB-binding in according to one embodiment of the present invention;

FIG. 8B illustrates the UB-binding activities of A. thaliana OTU2 N- and C-terminal variants of FIG. 8A;

FIG. 9A is a schematic diagram illustrating the deletion variants of OTLD1 that were used to delineate the domains and residues involved in UB-binding in according to one embodiment of the present invention;

FIG. 9B illustrates the relative UB-binding activities of A. thaliana OTLD1 N- and C-terminal variants of FIG. 9A in according to one embodiment of the present invention;

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### DEFINITIONS

The terms “polypeptide” and “protein” are used interchangeably herein and refer to amino acids in a polymeric form of any length, linked together by peptide bonds.

The term “nucleic acid(s)” and “polynucleotide(s)” are used interchangeably herein to mean nucleotides, either ribonucleotides or deoxyribonucleotides or a combination of both, in a polymeric unbranched form of any length.

The term “identical” or “percent (%) identity” as used herein refers to two or more sequences or subsequences that are the same or have a specified percentage of amino acid residues or nucleotides that are the same, when compared and aligned for maximum correspondence. To determine the percent identity, the sequences are aligned for optimal comparison purposes (e.g., gaps can be introduced in the sequence of a first amino acid sequence for optimal alignment with a second amino acid sequence). The amino acid residues or nucleotides at corresponding positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In certain embodiments, the two sequences are the same length. Methods for alignment of sequences for comparison are well known in the art, such methods include GAP, BESTFIT, BLAST, FASTA and TFASTA.

The term “ubiquitin (UB)” as used herein is interpreted broadly to encompass the full length UB (i.e., 76 amino acids in length) and its fragment, as long as it is active as a substrate for isopeptidase (e.g., the isolated polypeptide of the present disclosure). Preferably, the UB fragment in the present disclosure comprises at least 25%, 30%, 40%, 50%, 60%, 70%, 75%, 80%, 85%, or more particularly, 90%, 95%, 97%, 99% or more of the full length UB. Throughout this specification, the term “a UB chain” refers to a UB polymer composed of at least 3, 4, 5, 6, 7, 8, 9, 10, 11, 12, 13, 14, 15, 16, 17, 18, 19, or 20 units of UBs; preferably at least 5, 6, 7, 8, 9, 10, 11, 12, 13, 14, 15, 16, 17, 18, 19, or 20 units of UBs. When the distal or proximal UB is used, it is in relative to its proximity to a substrate protein, for example, the distal UB refers to the UB of a UB chain that is away from the substrate protein, whereas the proximal UB refers to the UB of a UB chain that is close to the substrate protein (see Komander and Rape, (2012) Annu. Rev. Biochem. 81, 203-229).

The term “plant” as used herein encompasses whole plants, ancestors and progeny of the plants and plant parts, including seeds, shoots, stems, leaves, roots, flowers, tissues and organs. The term “plant” also encompasses plant cells, suspension cultures, callus tissue, embryos, meristematic regions, gametophytes, sporophytes, pollen and microspores.

The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The present disclosure is based, at least in part, unexpected discovery of several novel ovarian tumor (OTU) domain containing polypeptides identified and isolated from A. thaliana respectively possess deubiquitination (DUB) activity in vitro; hence these novel isolated OTU DUBs and fragments thereof are useful in methods and/or kits involving deubiquitination of a substrate protein.

Identifying Novel A. thaliana Polypeptide with Isopeptidase Activity

Accordingly, one aspect of the present disclosure is to provide novel polypeptides and/or fragments thereof isolated from A. thaliana. Each of the isolated polypeptide comprises an amino acid sequence at least 85% identical to any of SEQ ID NOs: 1 to 9; which is coded by a nucleic acid at least 90% identical to any of SEQ ID NOs: 10 to 18.

The novel isolated polypeptides and/or fragments thereof of the present disclosure typically possess at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any of SEQ ID NO: 1 to 9 from A. thaliana. Preferably, the novel isolated polypeptides and/or fragments thereof of the present disclosure typically possess at least 85%, 86%, 87%, 88%, 89%, 90%, 91%, 92%, 93%, 94%, 95%, 96%, 97%, 98%, 99% or 100% sequence identity to any of SEQ ID NO: 1 to 9 from A. thaliana.

According to embodiments of the present disclosure, each of the isolated polypeptide and/or fragments thereof possesses deubiquitination (DUB) activity in vitro, meaning it is capable of cleaving a peptide linkage, particularly an isopeptide linkage between two UBs, such as the isopeptide located between the C-terminus of the distal UB and any of the first methionine (M1), the 48^{th} (K48) or the 63^{rd} lysine (K63) of the proximal UB in a UB chain, in which the UB chain may be optionally conjugated with a substrate protein.

Nucleic acids encoding the isolated polypeptides and/or fragments thereof of the present disclosure are represented by polynucleotide sequences that is any of SEQ ID NO: 10 to 18. Nucleic acids encoding the isolated polypeptides and/or fragments thereof of the present disclosure need not to be the full length nucleic acids. According to some embodiments of the present disclosure, a portion of any of the SEQ ID NO: 10 to 18 or a nucleic acid at least 90%, 91%, 92%, 93%, 94%, 95%, 96%, 97%, 98%, 99% or 100% identical to SEQ ID NO: 10 to 18, capable of expressing an amino acid sequence at least 85% identical to any of SEQ ID NO: 1 to 9, is sufficient to carry out the present invention. A portion of nucleic acid may be prepared by making one or more deletion to the nucleic acid, which may be used in its isolated form to produce the isolated polypeptides and/or fragments thereof of the present disclosure. Furthermore, nucleic acid variants may also be obtained by site-directed mutagenesis. Several methods are available for site-directed mutagenesis, in which PCR-based method is the most commonly adopted one.

In one example, the isolated polypeptide has the amino acid sequence of SEQ ID NO: 1, which is encoded by the nucleic acid of SEQ ID NO: 10; and mutations at amino acid residues 92, 89 and 288 of this isolated polypeptide abolish its activity to cleave the peptide linkage between the C-terminus of the distal UB and the M1, K48, or K63 of the proximal UB of the UB chain.

In other embodiments, the isolated polypeptide has the amino acid sequence of SEQ ID NO: 2, which is encoded by a nucleic acid of SEQ ID NO: 11, and two mutations at amino acid residues 63 and 99, or 179 and 195 of the isolated polypeptide, abolish its activity to bind ubiquitin.

In another example, the isolated polypeptide has the amino acid sequence of SEQ ID NO: 4, which is encoded by the nucleic acid of SEQ ID NO: 13, and a mutation at amino acid residue 136 of this isolated polypeptide abolish its activity to cleave the peptide linkage between the C-terminus of the distal UB and K48 or K63 of the proximal UB in the UB chain. In one preferred example, the isolated polypeptide has the amino acid sequence of SEQ ID NO: 5, which is encoded by the nucleic acid of SEQ ID NO: 14.

In still another example, the isolated polypeptide has the amino acid sequence of SEQ ID NO: 6, which is encoded by the nucleic acid of SEQ ID NO: 15, and a mutation at amino acid residue 48 of this isolated polypeptide abolish its activity to cleave the peptide linkage located between the C-terminus of the distal UB and K48 or K63 of the proximal UB in the UB chain.

In a further example, the isolated polypeptide has the amino acid sequence of SEQ ID NO: 8 or 9, which is encoded by the nucleic acid of SEQ ID NO: 17 or 18, and this isolated polypeptide is capable of cleaving the peptide linkage between the C-terminus of the distal UB and K63 of the proximal UB in the UB chain, at about pH 9 to 9.5.

According to some embodiments of the present disclosure, the isolated polypeptide has the amino acid sequence of any of SEQ ID NO: 1, SEQ ID NO: 3, SEQ ID NO: 4, SEQ ID NO: 6, SEQ ID NO: 8, or SEQ ID NO: 9; and is capable of cleaving the peptide linkage between the C-terminus of the distal UB and K48 or 63 of the proximal UB in the UB chain, at about pH 7 to 8.

Methods and Kits for Deubiquitinizing A Substrate Protein

The afore-identified and isolated A. thaliana polypeptides and/or fragments thereof may be packaged into kits for deubiquitinating a substrate protein.

In some embodiments, the kits comprise at least one of the isolated A. thaliana polypeptides and/or fragments thereof described above. The kits may optionally comprise one or more isopeptidase or deubiquitinase that is different from the isolated A. thaliana polypeptides and/or fragments thereof of the present disclosure; and instructions that explain how to conduct the deubiqutination assay. Other optional reagents in the kit may include appropriate buffer solutions for performing isopeptidase reaction. As used herein, “instruction” includes a publication, a recording, a diagram, or any other medium of expression that can be used to communicate or teach the user how to perform a method of the present invention. The instruction can be affixed to a container, which contains a kit of the invention. Alternatively, the instruction is packed independently from the container that comprises the kit of the invention.

It is therefore a further aspect of the present disclosure to provide a method of deubiquitinating a substrate protein by use of the kit of the present disclosure.

The substrate protein may be any protein modified with at least one UB and/or its fragment. The substrate protein may be isolated from a biological sample, which includes, but is not limited to, a plant sample, and an animal sample. In one preferred embodiment, the biological sample suitable for use in the present method is a plant sample, which includes, but is not limited to, a plant tissue sample, a plant cell sample, and a biological plant fluid sample.

The method includes the step of, subjecting the substrate protein having a conjugated UB chain to the action of the isolated polypeptide and/or fragment thereof of the present disclosure for a sufficient period of time, so as to allow the cleavage of the peptide linkage between the C-terminus of the distal UB and any of the first methionine (M1), the 48^{th} (K48) or the 63^{rd} lysine (K63) of the proximal UB in the UB chain, wherein the isolated polypeptide has an amino acid sequence at least 85% identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

In one example, the method includes the step of incubating the substrate protein with an isolated polypeptide having an amino acid sequence at least 85% identical to SEQ ID NO: 1 for a sufficient period of time, until the isopeptide linkage between the C-terminus of the distal UB and the M1, K48, or K63 of the proximal UB in the UB chain is cleaved.

In another example, the method includes the step of incubating the substrate protein with an isolated polypeptide having an amino acid sequence at least 85% identical to SEQ ID NO: 5 for a sufficient period of time, until the peptide linkage between the C-terminus of the distal UB and the K48 or K63 of the proximal UB in the UB chain is cleaved.

In still another example, the method includes the step of incubating the substrate protein with an isolated polypeptide having an amino acid sequence at least 90% identical to SEQ ID NO: 6, for a sufficient period of time, until the peptide linkage between the C-terminus of the distal UB and the K48 or K63 of the proximal UB in the UB chain is cleaved.

In a further example, the method includes the step of incubating the substrate protein with an isolated polypeptide having an amino acid sequence at least 90% identical to SEQ ID NO: 8 or 9, for a sufficient period of time, until the isopeptide linkage between the C-terminus of the distal UB and the K48 or K63 of the proximal UB in the UB chain is cleaved, which occurs at about pH 9 to 9.5.

According to some embodiments of the present disclosure, the method includes the step of incubating the substrate protein having a UB chain conjugated thereto with an isolated polypeptide having an amino acid sequence at least 85% identical to any of SEQ ID NO: 1 (OTU1), SEQ ID NO: 3 (OTU3), SEQ ID NO: 4 (OTU4c), SEQ ID NO: 6 (OTU7a), SEQ ID NO: 8 (OTU9), or SEQ ID NO: 9 (OTU10), for a sufficient period of time, until the isopeptide linkage between the C-terminus of the distal UB and K48 or K63 of the proximal UB in the UB chain is cleaved, which occurs at about pH 7 to 8.

In other embodiments, the method includes the step of incubating the isolated polypeptide having the amino acid sequence at least 85% identical to SEQ ID NO: 2, for a sufficient period of time, until the isopeptide linkage between the C-terminus of the distal UB and the K48 or K63 of the proximal UB in the UB chain is cleaved.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

Bioinformatics

The A. thaliana and O. sativa OTU loci were identified by reiterative searches of the databases respectively maintained by Arabidopsis Information Resource (TAIR) (Phoenix Bioinformatics, Redwood City, Calif., U.S.A.) and National Center for Biotechnology Information (NCBI) (Bethesda, Md., USA) using the H. sapiens OTUB1 and OTUB2 and S. cerevisiae Otu1 sequences as initial queries. Protein domains were identified with SMART from the ExPASy server. Routine DNA and protein sequence analyses were conducted with GCG Version 11.1.3-UNIX (Accelrys Inc.).

To generate the phylogenetic tree, the OTU domain sequences were aligned using MUSCLE with the default settings. The resulting alignment was used to infer the phylogeny by using maximum likelihood and Bayesian methods. We used the PhyML program for the maximum likelihood method (Guindon and Gascuel, (2003) Syst. Biol. 52, 696-704.). To estimate the level of support for each internal branch, we generated 1,000 non-parametric bootstrap samples of the alignment by using the SEQBOOT program from the PHYLIP package (Felsenstein, (1989) Cladistics 5, 164-166.) and repeated the phylogenetic inference as described above. For the Bayesian approach, we used the program MrBayes (Ronquist and Huelsenbeck, (2003) Bioinformatics 19, 1572-1574; Altekar et al., (2004) Bioinformatics 20, 407-415.) to infer the posterior probability distributions.

The accession numbers for OTU-containing sequences from O. sativa, H. sapiens, and S. cerevisiae with abbreviated binominal name prefixes are as follows: AK120577 (Os01g0900900), EAY84610 (Os02g06890), BAD38558 (Os02g31830), BAD26139 (Os02g32180), EEC73285 (Os02g32190), BAD26147 (Os02g32280), AK067291 (Os02g0168600), NP_{—}001046946 (Os02g0513800), AK119352 (Os02g0819500), NP_{—}001049654 (Os03g0266000), AK072986 (Os03g0589300), AK071971 (Os03g0859800), AK240901 (Os04g32970), AK101471 (Os04g0414100), AK066247 (Os04g0619500), AK107489 (Os04g0652600), AK103099 (Os04g0670400), AK073551 (Os06g0669800), EEE69074 (Os08g42540), NP_{—}001062186 (Os08g0506000), NP_{—}001063527 (Os09g0487700), Q9NP73 (HsALG13), NP_{—}060140 (HsOTUB1), NP_{—}075601 (HsOTUB2), NP_{—}001138845 (HsOTUD1), Q5T2D3 (HsOTUD3), EAX05048 (HsOTUD4), NP_{—}060072 (HsOTUD5), Q7L8S5 (HsOTUD6A), AAH29760 (HsOTUD6B), NP_{—}570971 (HsOTUD7A), NP_{—}064590 (HsOTUD7B), Q96BN8 (HsOTULIN/FAM105B), NP_{—}006281 (HsTNFAIP3/A20), NP_{—}079330 (HsVCPIP1), NP_{—}061036 (HsYOD1), CAB64449 (HsZRANB1), P43558 (ScOTU1), and P38747 (ScOTU2).

Recombinant OTU Protein Purification

To express various His- and GST-tagged OTU proteins of the present disclosure, corresponding full-length coding regions were PCR-amplified using PfuTurbo (Agilent Technologies) and cloned in frame into pET28 or pET42 vectors, respectively (EMD Millipore). Specific primers for cloning the OTU proteins, their site-specific variants are listed in Table 1. Mutagenesis was performed with PfuTurbo according to manufacturer's instructions (Agilent Technologies). The sequences of all expression constructions were verified as correct by DNA sequence analysis with an ABI PRISM 3700 DNA Analyzer (Life Technologies). Recombinant protein expression in E. coli BL21 (DE3) (Novagen) and purification has been previously described (Fatimababy et al., (2010) FEBS J. 277, 796-816).

TABLE 1
Primers for the expression of A. thaliana OTU-DUBs and their site-specific variants

| | Locus or | | SEQ |
|---|---|---|---|
| | accession/ | | ID |
| Name | Template | PCR primers for amplification or mutagenesis | NO. |
| OTU1 | At2g28120/ CD4-14 | 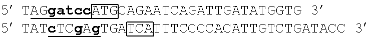 | 19 20 |
| OTU1-D8 | At2g28120/ | 5′ GGCGAACAAGAGGAGAaGGAAATTGCTTCTTCC 3′ | 21 |
| 9E | OTU1 | 5′ GGAAGAAGCAATTTCCtTCTCCTCTTGTTCGCC 3′ | 22 |
| OTU1-C92 | At2g28120/ | 5′ CAAGAGGAGATGGAAATaGCTTCTTCCGAAGTTTTATG 3′ | 23 |
| S | OTU1 | 5′ CATAAAACTTCGGAAGAAGCtATTTCCATCTCCTCTTG 3′ | 24 |
| OTU1-H2 | At2g28120/ | 5′ CTGTATCGTCCAGGCCgTTACGATATCCTCTAC 3′ | 25 |
| 88R | OTU1 | 5′ GTAGAGGATATCGTAAcGGCCTGGACGATACAG 3′ | 26 |
| GST-OTU1 | At2g28120/ | Mobilized from OTU1 | |
| | OTU1 | | |
| OTU2 | At1g50670/ CD4-12 | 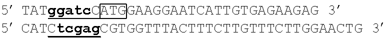 | 27 28 |
| GST-OTU2 | At1g50670/ | Mobilized from OTU2 | |
| | OTU2 | | |
| OTU3 | At2g38025/ CD4-12 | 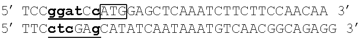 | 29 30 |
| GST-OTU3 | At2g38025/ | Mobilized from OTU3 | |
| | OTU3 | | |
| OTU4a | At3g57810/ CD4-14 | 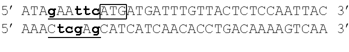 | 31 32 |
| OTU4c | At3g57810/ CD4-14 | 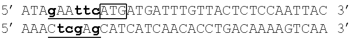 | 33 34 |
| OTU4d | At3g57810/ CD4-14 | 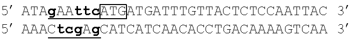 | 35 36 |
| GST-OTU4c | At3g57810/ | Mobilized from OTU4c | |
| | OTU4c | | |
| GST-OTU4c- | At3g57810/ | 5′ CCGGGGGATGGCAGAaGTTTGTTTCGTTCTGTAG 3′ | 37 |
| C136S | GST-OTU4c | 5′ CTACAGAACGAAACAAACtTCTGCCATCCCCCGG 3′ | 38 |
| OTU5a | At3g62940/ CD4-14 | 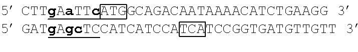 | 39 40 |
| OTU5b | At3g62940/ CD4-14 | 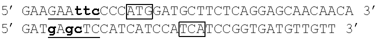 | 41 42 |
| GST-OTU5a | At3g62940/ | Mobilized from OTU5a | |
| | OTU5a | | |
| GST-OTU5b | At3g62940/ | Mobilized from OTU5b | |
| | OTU5b | | |
| OTLD1Aa | At2g27350/ | Mobilized from GST-OTLD1Aa | |
| | GST-OTLD | | |
| | 1Aa | | |
| GST-OTL D1Aa | At2g27350/ CD4-14 | 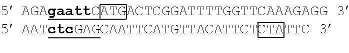 | 43 44 |
| OTU7a | At5g67170/ | 5′ GATggatccATGGCGAAAACTAAACAACAGAAATCTAGG 3′ | 45 |
| | CD4-14 | 5′ ATACTcdAgTTATGTCTTCGCCTTGAGTGAGTGAGAC 3′ | 46 |
| OTU7a-C4 | At5g67170/ | 5′ GTGACTGCTGACGGTAATaGTTTCTTCAGAGCAATTGCTG 3′ | 47 |
| 8S | OTU7a | 5′ CAGCAATTGCTCTGAAGAAACtATTACCGTCAGCAGTCAC 3′ | 48 |
| GST-OTU7a | At5g67170/ | Mobilized from OTU7a | |
| | OTU7a | | |
| OTU9 | At5g04250/ CD4-14 | 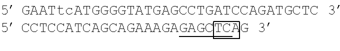 | 49 50 |
| GST-OTU9 | At5g04250/ | Mobilized from OTU9 | |
| | OTU9 | | |
| OTU10 | At5g03330/ CD4-14 | 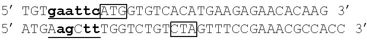 | 51 52 |
| GST-OTU10 | At5g03330/ | Mobilized from OTU10 | |
| | OTU10 | | |
| OTU11a | At3g22260/ CD4-14 | 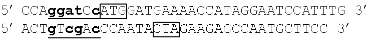 | 53 54 |
| GST-OTU11a | At3g22260/ | Mobilized from OTU11a | |
| | OTU11a | | |
| OTU12 | At3g02070/ CD4-14 | 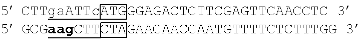 | 55 56 |
| GST-OTU12 | At3g02070/ | Mobilized from OTU12 | |
| | OTU12 | | |
| HsOTUB1 | AK000120/ 1^{st} strand cDNAs | 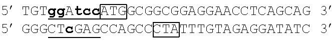 | 57 58 |
| UB2 | JX089332/ CD4-22 | 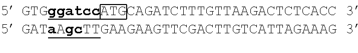 | 59 60 |
| UB3 | JX089333/ CD4-22 | 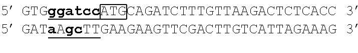 | 61 62 |
| UB4 | JX089331/ CD4-22 | 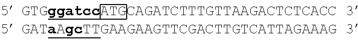 | 63 64 |
| RAD23d-1 | At5g38470/ CD4-14 | 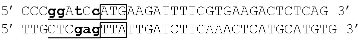 | 65 66 |
| RAD23d-2 | At5g38470/ RAD23d-1 | 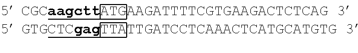 | 67 68 |
| GST | /pET42a | 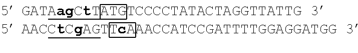 | 69 70 |
| HA3-UBQ | 1538658/UB2 | 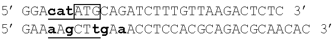 | 71 72 |
| HA3-UBQ- | 1538658/HA | The NcoI-Hind III fragment containing the HA3-tagged RUB | |
| GST | 3-RUB-GST | fragment in HA3-RUB-GST was replaced by the HA3-UBQ | |
| | | fragment from HA3-UBQ | |
| RUB | At1g31340/ CD4-13 | 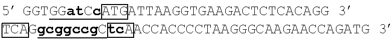 | 73 74 |
| HA3-RUB-1 | At1g31340/ RUB | 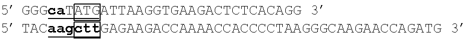 | 75 76 |
| HA3-RUB- | At1g31340/ | The NcoI-Hind III fragment containing the HA3-tagged RUB | |
| 2 | HA3-RUB-1 | from HA3-RUB-1 was mobilized into RAD23d-2 | |
| HA3-RUB- | At1g31340/ | The HindIII-XhoI fragment containing RAD23d in HA3-RUB-2 | |
| GST | HA3-RUB2 | was replaced by the similar restricted fragment from GST | |
| HA3-SUMO- 1 | At4g26840/ CD4-22 | 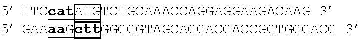 | 77 78 |
| HA3-SUMO- | At4g26840/ | The NcoI-Hind III fragment containing the HA3-tagged SUMO | |
| 2 | HA3-SUMO-1 | from HA3-SUMO-1 was mobilized into RAD23d-2 | |
| HA3-SUMO- | At4g26840/ | The HindIII-XhoI fragment containing RAD23d in | |
| GST | HA3-SUMO- | HA3-SUMO-2 was replaced by the similar restricted fragment | |
| | 2 | from GST | |

Note:
Bases in upper case and plain text are identical to their templates, and bases in lower case and bold-face are mutated compared to their templates. Underlined bases are native or engineered restriction sites. Boxed in 5′ primer are ATG start codons. Boxed in 3′ primers are native or engineered (those with lower case/bold letters) stop codons. Boxed in SEQ ID NOs: 76, and 78 are mutated native stop codons for in frame fusion to HA tag.

In Vitro Deubiquitylation Assay

The in vitro deubiquitylation assay was performed as previously described (Balakirev et al., (2003) EMBO Rep. 4, 517-522). The purified recombinant OTU proteins and their variants of 300 nM each, along with different substrates, were incubated in the reaction buffer (150 mM NaCl, 0.5 mM DTT, and 20 mM Tris-HCl, pH 8) at 37° C. for 1 h. For optima pH, cleavage reactions were performed in buffers containing 150 mM NaCl and 0.5 mM DDT at different pH (20 mM acetate buffer at pH 4.5, 20 mM phosphate buffer at pH 6.5, 20 mM Tris-HCl buffer for pH 7.5-8.5 or 20 mM 3-cyclohexylamino-1-propanesulfonic acid for pH 9.5-10.5). The hydrolysis assay was performed at 37° C. for 20 min. The reactions were stopped by boiling for 5 min in 2×SDS-PAGE sample buffer. The inputs and cleavage products were separated by SDS-PAGE and monitored by immunoblotting with rabbit polyclonal antisera raised against H. sapiens UB (sc-9133) or mouse monoclonal antisera raised against the influenza hemagglutinin (HA) epitope (sc-7392) (Santa Cruz Biotechnology). The input protein concentrations were determined with the protein assay reagent (Bio-Rad Laboratories). The substrates were used in quantities of 250 ng for the K48- and K63-tetraubiquitin chains (Boston Biochem), 100, 200, and 250 ng for the purified recombinant linear UB dimers, trimers, and tetramers, respectively, and 250 ng for the purified recombinant HA-tagged UB, RUB, and SUMO fusions.

UB Chain Binding

The GST pull-down assay and the detection of pulled-down products by immunoblotting has been previously described (Fatimababy et al., (2010) FEBS J. 277, 796-816; Lin et al., (2011) Plant Cell 23, 2754-2773). The Lys48- and Lys63-linked UB chains (Ub2-7) were purchased from Boston Biochem.

### Example 1

### A. thaliana has an OTU-DUB Family with 12 Phylogenetically Distinct Members

To characterize A. thaliana OTU-DUBs, 12 loci OTU1-5, OTLD1, and OTU7-12 encoding OTU domain-containing proteins were identified by database searches using the OTU domain sequences of H. sapiens OTUB1 (NP_{—}060140) and OTUB2 (NP_{—}075601) and S. cerevisiae Otu1 (P43558) as initial queries. The exon-intron organization and coding sequences of these A. thaliana loci were determined by sequence comparison with corresponding PCR-amplified full-length cDNAs and available cDNAs and ESTs from the TAIR database. Based on the detected splicing products, a single isoform exists for each of the OTU1-3, OTU9-10, and OTU12 loci, two isoforms exist for each of the OTU5, OTLD1, OTU7, and OTU11 loci (designated with a and b extensions), and five isoforms exist for OTU4 (OTU4a-e). With the exception of OTU4b and OTU4d-e, in which the encoded proteins were predicted to have their OTU domains disrupted or lost as a result of frame shifts caused by alternative splicing, the rest of the OTU loci encode potential OTU domain-containing DUBs. However, OTU8 is likely a pseudogene because corresponding multiple isolated cDNAs were consistently derived from an alternative splicing using a different 3′ junction (61 by upstream of the predicted one) of the second annotated intron, which generated a frame-shift and a downstream start codon located on the third annotated exon, resulting in an N-terminally truncated OTU domain. In agreement with this idea, a search of GENEVESTIGATOR microarray databases showed extremely low expression levels for OTU8 transcripts and moderate to high expression levels for OTU1-2, OTU4-5, OTLD1, OTU9, and OTU11-12 transcripts across various primary and cultured tissues from different organ sources (data not shown). Microarray probes are not available for OTU3, OTU7, and OTU10. In general, constitutive expression of the eleven active OTU loci in various tissues, including roots, inflorescence stems, rosette and cauline leaves, flowers, and siliques, was detected by RT-PCR (data not shown).

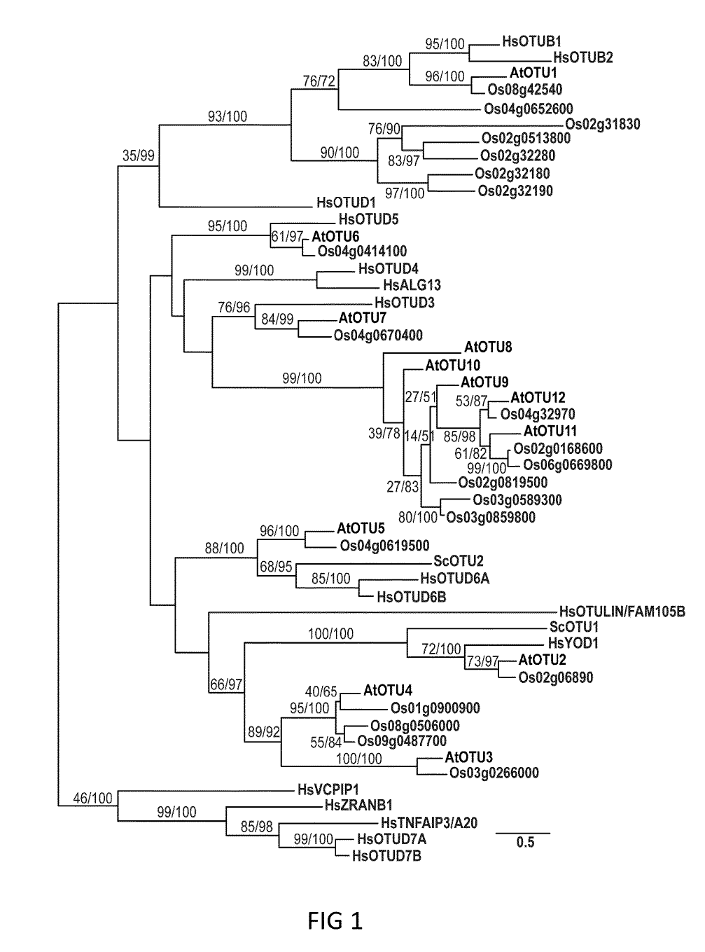

To examine the phylogenetic relationship of the 12 potential A. thaliana OTU-DUBs as described above, a phylogenetic tree (FIG. 1) was generated based on the aligned peptide sequences from the OTU domains derived from A. thaliana, Oryza sativa, H. sapiens, and S. cerevisiae OTU proteins. With the exception of OTU8-12, which reside in the same clade, the rest of the A. thaliana OTU proteins, each belongs to a distinct clade in the deduced phylogenetic tree. A single potential O. sativa ortholog is associated with each of the A. thaliana OTU2, OTU3, OTU5, OTLD1, and OTU-7. However, multiple potential O. sativa orthologs are associated with the clades containing A. thaliana OTU1, OTU4, and OTU9. The closer phylogenetic relationship among A. thaliana and O. sativa OTU-orthologs within the same clades compared with OTU proteins of the same species from different clades indicates that the orthologs within a clade were evolved before the separation of dicot and monocot species. Among A. thaliana and O. sativa orthologs within the same clade, the near identical exon/intron organization of their corresponding loci further supports their close phylogenetic relationship. Five clades harbor OTU proteins derived from A. thaliana, O. sativa, and H. sapiens, of which the two clades containing A. thaliana OTU2 and OTU5 also harbor S. cerevisiae Otu1 and Otu2, respectively. The five cross-species conserved clades appear to have evolved at times earlier than the divergence of their common ancestors. The A. thaliana OTU1, OTU2, OTU5, OTLD1, and OTU7 belong to the same clade of H. sapiens OTUB1/OTUB2, H. sapiens YOD1/S. cerevisiae Otu1, H. sapiens OTUD6A/OTUD6B/S. cerevisiae Otu2, H. sapiens OTUD5, and H. sapiens OTUD3 respectively, suggesting that these A. thaliana OTU proteins carry distinct evolutionarily conserved functions. No plant orthologs were associated with four H. sapiens-specific clades including OTUD1, OTUD4/ALG13, the arbitrary outgroup clade harboring VCPIP1, ZRANB1, TFNAIP3/A20, OTUD7A, and OTUD7B, and also the OTULIN/FAM105B clade. Similarly, three plant-specific clades harboring A. thaliana OTU3, OTU4, and OTU8-12, each with one or multiple O. sativa homologs, were observed.

### Example 2

### A. thaliana OTU-DUBs Exhibit Distinct Substrate Specificities

The lengths of encoded A. thaliana OTU proteins ranged from 208 to 506 amino acids, with predicted molecular weights of about 23.4-55.3 kDa (Table 2). Each protein carries an OTU domain (PFAM accession number: PF02338) that is 113-203 amino acids in length. The A. thaliana OTU1, similar to its H. sapiens counterparts OTUB1 and OTUB2, carries a larger Peptidase_C65 domain (PF10275) to cover the OTU domain. The OTU domain is located at the center of OTLD1, at C-terminal regions for OTU1, OTU3-5, and OTU9-12, and at N-terminal parts of OTU2 and OTU7. All of the detected A. thaliana OTU domains carry the conserved catalytic Cys-His-Asp/Asn triad similar to known active OTU-DUBs, such as H. sapiens OTUB1 (data not shown). In addition to the OTU domain, a few known domains were identified to associate with the A. thaliana OTU proteins including a coiled-coil domain in OTU5a (19-86) and OTU5b (4-71), a SCOP UBA-like domain (d1fya) in OTLD1a (453-491) and OTLD1b (452-490), and a potential nucleic acid binding SEC-C motif (PF02810) in OTU7a (313-333) (Table 2).

TABLE 2
Identified A. thaliana Loci Encoding OTU-DUBs

| | | Peptide | | |
|---|---|---|---|---|
| | | length | Identified | Full-length |
| | | (aa)/MW | domains | cDNA |
| Gene | Locus | (kDa) | (coordinates)^{a} | accession^{b} |
| OTU1 | At1g28120 | 306/34.4 | Peptidase_C65 | JQ013442 |
| | | | (41-294) | |
| | | | OTU (87-289) | |
| OTU2 | At1g50670 | 208/23.4 | OTU (11-123) | JQ013443 |
| OTU3 | At2g38025 | 234/26.3 | OTU (82-230) | JQ013444 |
| OTU4a | At3g57810 | 317/35.8 | OTU (174-300) | JQ013445 |
| OTU4b | | 217/24.2 | OTU* (174-216) | BX824976 |
| OTU4c | | 274/30.8 | OTU (131-257) | JQ013446 |
| OTU4d | | 195/21.8 | OTU* (131-191) | JQ013447 |
| OTU4e | | 75/8.3 | ND | JQ013448 |
| OTU5a | At3g62940 | 332/37.4 | OTU (187-321) | JQ013449 |
| | | | Coiled coil (19-86) | |
| OTU5b | | 316/35.7 | OTU (172-306) | JQ013450 |
| | | | Coiled coil (4-71) | |
| OTLD1a | At2g27350 | 506/55.3 | OTU (222-333) | JQ013451 |
| | | | UBA-like (453-491) | |
| OTLD1b | | 505/55.3 | OTU (222-333) | AY058065 |
| | | | UBA-like (452-490) | |
| OTU7a | At5g67170 | 375/41.5 | OTU (43-155) | JQ013452 |
| | | | SEC-C (313-333) | |
| OTU7b | | 231/26.9 | OTU (43-155) | JQ013453 |
| OTU8 | At2g39320 | 154/18.3 | OTU* (1-62) | JQ013454 |
| | | | Coiled coil (85-150) | |
| OTU9 | At5g04250 | 345/39.2 | OTU (210-322) | JQ013455 |
| OTU10 | At5g03330 | 356/41.7 | OTU (219-331) | JQ013456 |
| OTU11a | At3g22260 | 245/28.2 | OTU (107-219) | JQ013457 |
| OTU11b | | 24027.7 | OTU (107-219) | JQ013458 |
| OTU12 | At3g02070 | 219/25.6 | OTU (85-197) | JQ013459 |

^{a}The encoded proteins for OTU4b, OTU4d, OTU4e, and OTU8, had disrupted (OTU*) or lost (ND) OTU domain due to frame shifts caused by alternative splicing.
^{b}With the exception of OTU4b and OTLD1b, which have full-length cDNAs available in the TAIR database, the full-length cDNAs, as for various His- or GST-tagged wild-type recombinant constructs, were PCR-isolated from various cDNA libraries (see Table 1).

The distinct phylogenetic relationship of the A. thaliana OTU proteins strongly suggests their functional differentiation. To further explore this possibility, the differences of their biochemical properties, including deubiquitylation substrate specificities and binding activities against K48- and K63-linked ubiquitin (UB) chains, were further characterized. For these purposes, purified recombinant wild-type, site-specific, and deletion variants of A. thaliana OTU proteins were used, which were expressed in Escherichia coli as His-tagged or His and glutathione S-transferase (GST)-double tagged versions. The deubiquitylation substrate specificities were examined for A. thaliana OTU proteins OTU1-3, OTU4c-d, OTU5b, OTLD1a, OTU7a, OTU9-10, OTU11a, and OTU12, and results are summarized in Table 3. With the exception of OTU4c and OTU10, which were examined using the GST-tagged versions because of low yields for their His-tagged forms, the His-tagged versions were analyzed. A GST-tagged OTU1 was also analyzed and compared with the His-tagged form to examine whether the fused large GST tag affects catalytic activity. The His-tagged OTU4d was analyzed with the expectation of a catalytic defect because of a truncated OTU domain. A His-tagged H. sapiens OTUB1 was analyzed as a control and for comparison. Other OTU isoforms were not analyzed for various reasons, such as low yields of both tagged recombinant proteins (OTU4a), small sequence variation with the analyzed isoforms (OTU5a, OTLD1 b and OTU11b), large C-terminal truncation (OTU7b), and lost or disrupted OTU domains (OTU4b, OTU4e, and OTU8).

TABLE 3
Distinct Biochemical Properties of A. thaliana OTU-containing Deubiquitinases

| | | Ub4 | | Linear chain | | | Ub/Ubl-GST fusion | | | |
|---|---|---|---|---|---|---|---|---|---|---|
| Name^{a} | Tag | K48 | K63 | Ub2 | Ub3 | Ub4 | Ub | Rub | Sumo | Chain^{b} |
| OTU1 | His | +++^{c} | + | +++ | +++ | +++ | − | − | − | − |
| OTU1 | GST | +++ | + | +++ | +++ | +++ | − | − | − | − |
| OTU2 | His | + | + | − | − | − | − | − | − | K48 ≈ |
| | | | | | | | | | | K63 |
| OTU3 | His | ++ | +++ | − | + | + | ++ | + | − | − |
| OTU4c | GST | +++ | +++ | − | ++ | ++ | ++ | + | − | − |
| OTU4d^{c} | His | − | − | − | − | − | − | − | − | − |
| OTU5b | His | − | − | − | − | − | − | − | − | − |
| OTLD1a | His | − | − | − | − | − | − | − | − | K48 > |
| | | | | | | | | | | K63 |
| OTU7a | His | + | ++ | − | − | − | ++ | + | − | − |
| OTU9 | His | ++ | ++ | − | − | − | ++ | + | − | − |
| TU10 | GST | ++ | +++ | − | − | − | ++ | + | − | − |
| OTU11a | His | − | − | − | − | − | − | − | − | − |
| OTU12 | His | − | − | − | − | − | − | − | − | − |
| HsOTUB1 | His | +++ | − | − | − | − | − | − | − | ND |

^{a}OTU4d contains a disrupted OTU domain.
^{b}Ubiquitin chain binding was analyzed by GST pull-down assays with GST fused proteins; −, absent of binding; ND, not determined.
^{c}The approximate deubiquitylation activities are quantitatively designated by +++, ++, +, and − for strong, moderate, weak, and absent of activity, respectively.

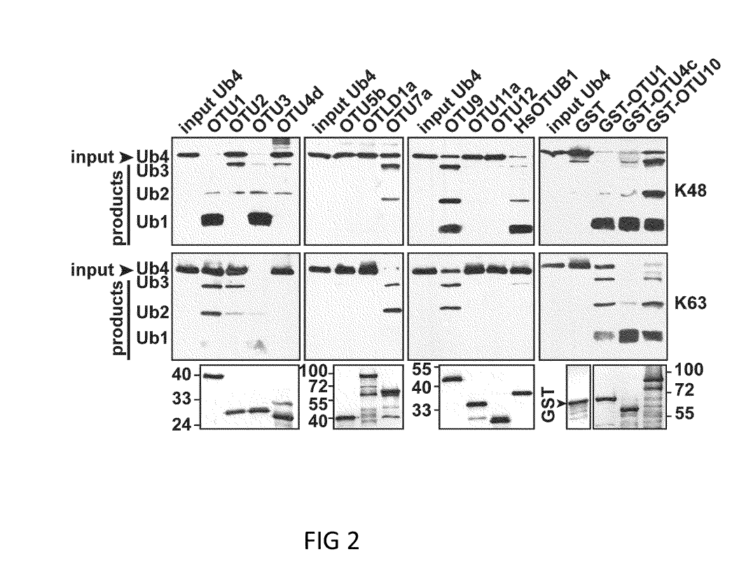

The substrate specificities of potential A. thaliana OTU DUBs were examined by determining their in vitro cleavage activities against isopeptide bond-linked UB tetramers of K48- and K63-linkage types, peptide bond-linked linear UB dimers, trimers, and tetramers, and peptide bond-linked UB-, RUB-, and SUMO-GST fusion proteins. As summarized in Table 3, seven of the eleven tested A. thaliana OTU proteins displayed deubiquitylation activities, with distinct substrate specificities. The selectivity of the potential A. thaliana OTU DUBs against the K48- and K63-linked UB chains was determined first (see Table 3; FIG. 2). The OTU1 of either the His- or GST-tagged version, similar to its H. sapiens counterpart OTUB1, showed a clear preference for K48-linked UB chains. However, unlike OTUB1, which barely displayed detectable activity against K63-linked chains, the A. thaliana OTU1 showed moderate activity with this chain type. By contrast, while OTU4c displayed a slight cleavage preference for the K63-linked UB chains, OTU3, OTU7a, and OTU10 had stronger cleavage activities against the K63-linked UB chains. However, OTU2 and OTU9 showed approximately similar cleavage activities for both linkage types. The rest of the examined A. thaliana OTU proteins, OTU5b, OTLD1a, OTU11a, and OTU12, did not display detectable in vitro cleavage activity against either chain-types or other tested substrates, suggesting their cryptic nature in vitro or specificity against other untested chain types. As predicted, the OTU4d with a disrupted OTU domain displayed no significant cleavage activity against either chain-types.

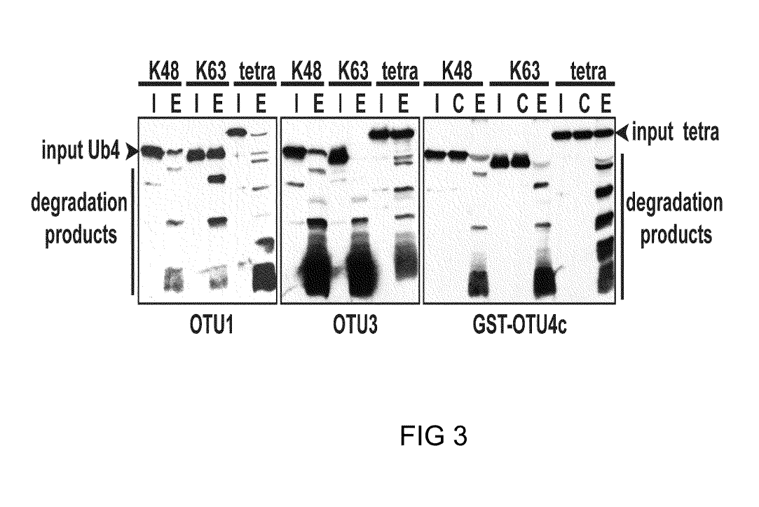

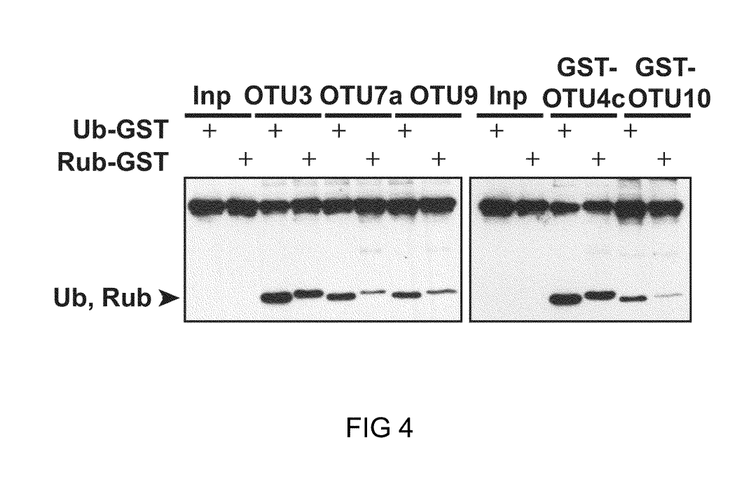

Among the seven A. thaliana OTU proteins active against isopeptide bond-linked UB chains, OTU1, OTU3, and OTU4c also displayed cleavage activities against peptide bond-linked linear UB polymers. OTU1 of both tagged forms displayed strong cleavage activities against all tested linear UB polymers, and OTU3 and OTU4 displayed weak to moderate cleavage activities against linear UB trimers and tetramers but not dimers (see Table 3; FIG. 3). Moreover, whereas A. thaliana OTU1 displayed similarly stronger catalytic activities against peptide bond- and K48 isopeptide bond-linked UB tetramers than that of K63-linked UB tetramers, OTU3 and OTU4c had stronger catalytic activities for isopeptide bond-linked UB tetramers of both linkage types than peptide bond-linked linear UB tetramers (FIG. 3). The A. thaliana OTU1 again displayed unique and strong catalytic activity against peptide bond-linked linear UB chains compared with H. sapiens OTUB1, which was inactive (Table 3). With the exception of OTU1 and OTU2, the active A. thaliana OTU proteins also displayed cleavage activities against peptide bond-linked UB- and RUB-GST fusions, but not the SUMO-GST fusion (Table 3; FIG. 4). A cleavage preference was evident for UB-GST over RUB-GST (Table 3; FIG. 4).

Examined by cleavage activities of isopeptide-bond linked ubiquitin tetramers of K48 and K63 linkage types, OTU1, OTU3, OTU4c, OTU7a, OTU9, and OTU10 generally displayed optimum activities at neutral pH, i.e., pH 7 to 8. However, for cleavage of K63-linked chain, OTU9 and OTU10 showed optimum activities at slightly alkaline pH, i.e., pH 9 to 9.5. The neutral and slightly alkaline pH optimum suggests the active A. thaliana OTU DUBs examined likely are not vacuolar proteases.

### Example 3

### A. thaliana OTU-DUBs are Cysteine Proteases with a Conserved Catalytic Triad

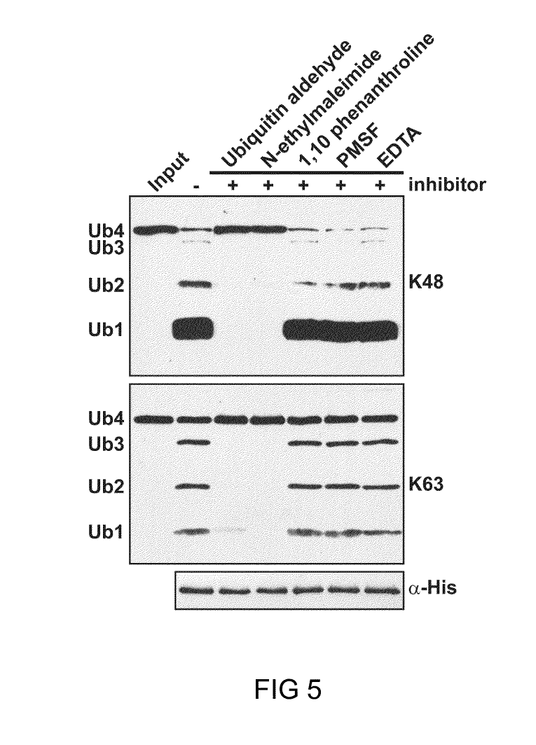

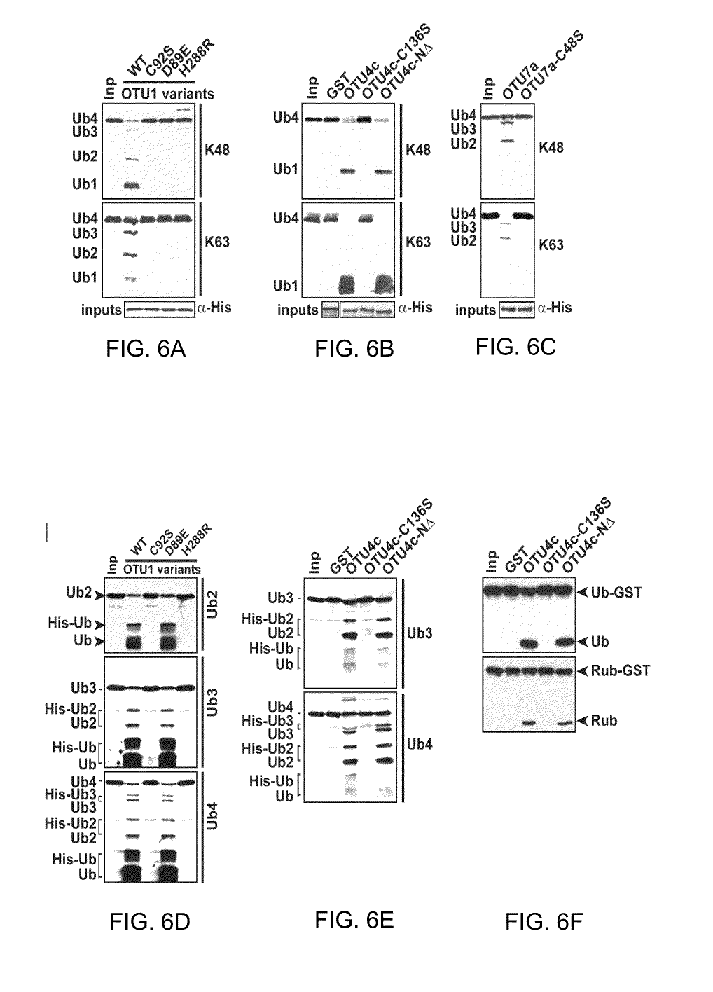

The presence of a conserved catalytic triad suggested that the A. thaliana OTU DUBs are cysteine proteases similar to those characterized from H. sapiens and S. cerevisiae. As tested with the His-tagged OTU1, the cleavage activities against K48- or K63-linked UB tetramers were inhibited by UB aldehyde and N-ethylmaleimide, but not by the metalloprotease inhibitors 1,10-phenanthroline and EDTA or the serine protease inhibitor phenylmethylsulfonyl fluoride (FIG. 5). Furthermore, site-specific mutations of the conserved catalytic triad in OTU1, OTU4c, and OTU7a or of a conserved residue that is potentially critical for stabilizing the oxyanion reaction intermediate in OTU1 (D89) completely abolished cleavage activity for K48- and K63-linked UB tetramers (FIGS. 6A to 6C, OTU1 variants C92S, D89E, and H288R, OTU4c-C136S, and OTU7a-C48S). The same mutations also generally abolished the linear UB polymer cleavage activities of OTU1 and OTU4c (FIGS. 6D and 6E) and the UB- and RUB-GST fusion protein cleavage activities for OTU4c (FIG. 6F). Interestingly, although cleavage of isopeptide bond-linked UB tetramers was abolished, OTU1-D89E was still cleaved linear UB polymers to a similar extent as the wild-type protein (FIG. 6D).

Further, an N-terminal 43-residues deletion in OTU4c did not modify its catalytic activities with the examined isopeptide and peptide bond-linked substrate (FIGS. 6B, 6E, and 6F, OTU4c-NΔ). Similarly, the cleavage of K48- and K63-linked tetra ubiqutin chains by N-terminal deletion variants of OTU9 and OTU10 (i.e, OTU9-NΔ 1-100, OTU9-NΔ1-172, OTU10-NΔ1-125, and OTU10-NΔ1-173) containing intact OTU domains was comparable to that catalyzed by the corresponding wild-type proteins (data not shown).

### Example 4

### A. thaliana OTU2 and OTLD1 Bind UB Chains with Distinct Linkage Preferences

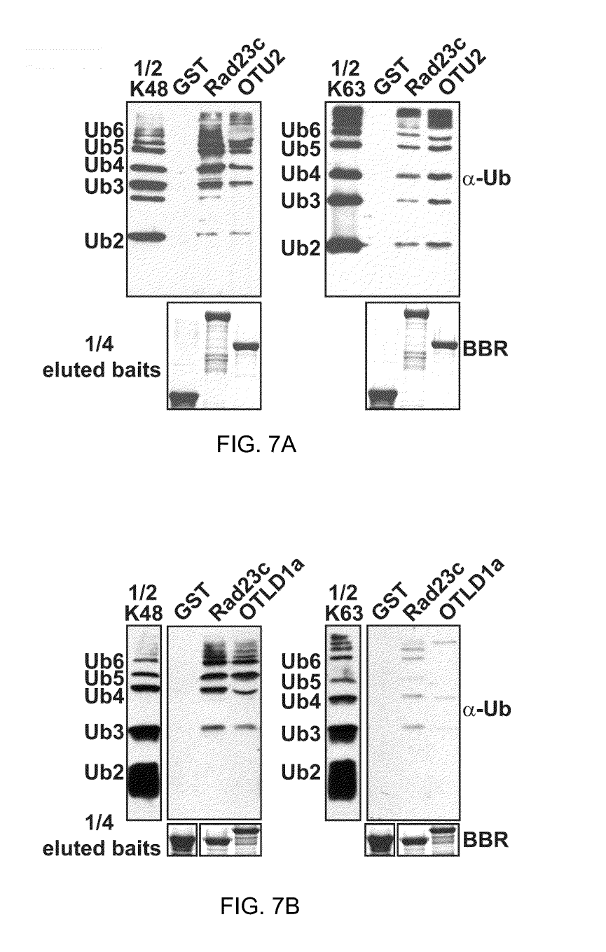

Because binding preferences for UB chains of different linkage types could potentially contribute to substrate selectivity or intracellular targeting, in vitro association of A. thaliana OTU-proteins with K48- and K63-linked UB chains by pull-down assays using their GST-fusions were investigated, and OTU1-3, OTU4c, OTU5b, OTLD1a, OTU7a, OTU9-10, OTU11a, and OTU12 were examined. However, the binding activities of K48- and K63-linked UB chains were only detected with OTU2 and OTLD1a (Table 3; FIGS. 7A and 7B). OTU2 displayed approximately equivalent binding affinities towards K48- and K63-linked UB chains, and OTLD1a showed a clear binding preference for K48-linked UB chains.

### Example 5

### OTU2 and OTLD1 Harbor Novel UB-Binding Domains

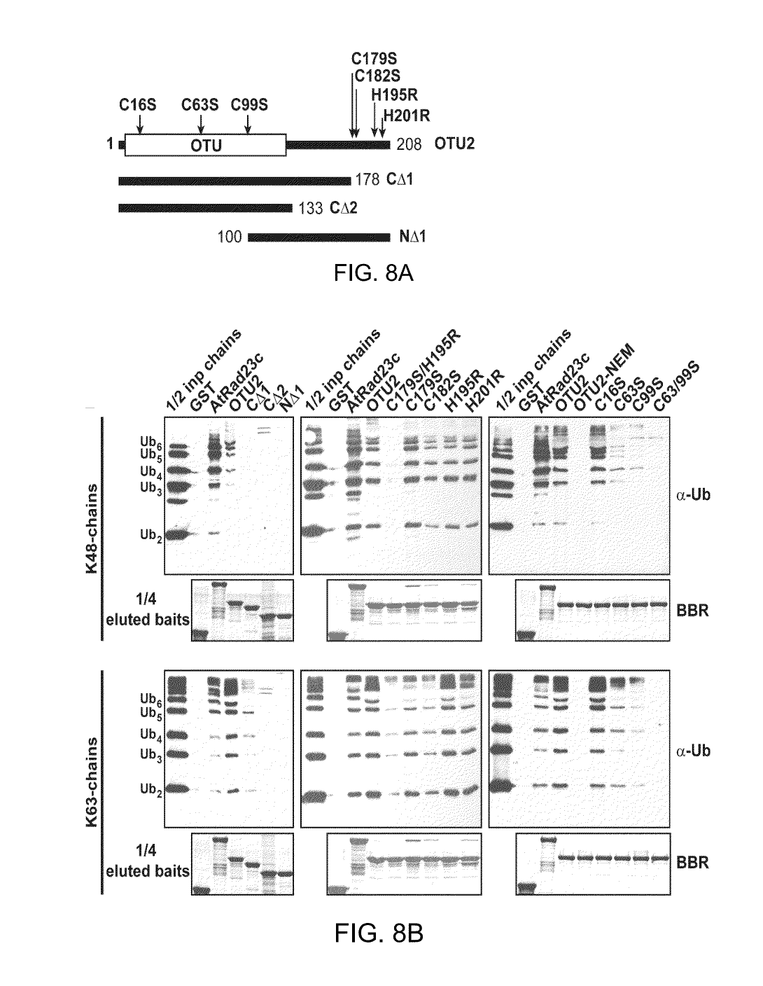

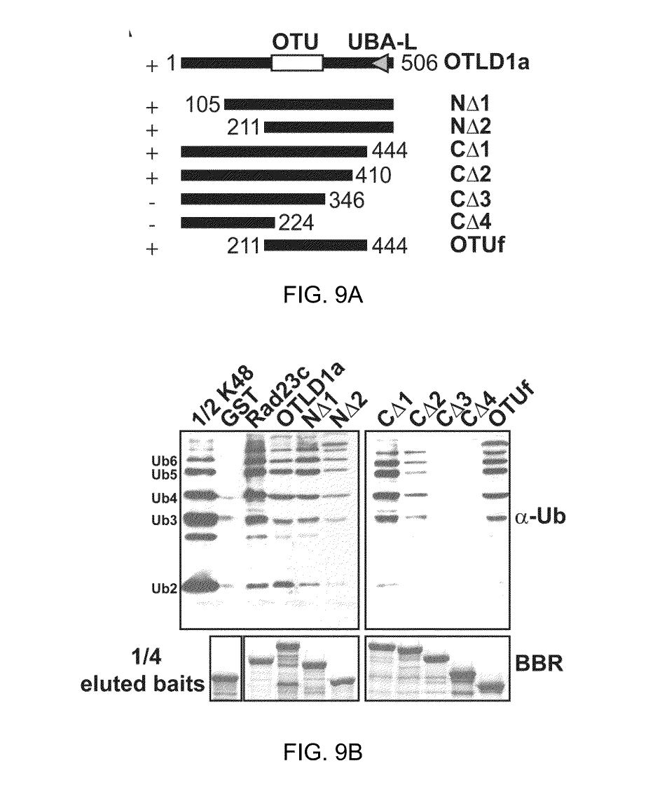

We delineated the involved critical domains or residues involved using GST-fused deletion and/or site-specific variants (FIGS. 8A and 9A). Binding assays using terminal truncations of OTU2 indicated both N-(1-99) and C-terminal parts (179-208) contain structural elements critical for binding K48- and K63-linked ubiquitin chains. The association with K48- and K63-linked ubiquitin chains was abolished for all OTU2 terminal deletion mutants (FIGS. 8A and 8B; CΔ1, CΔ2, and NΔ1). A PROSITE Zinc_Finger_C2H2 motif containing four well-spaced Cys and His residues was identified at OTU2 C-terminus (179-201). Individual mutants of the Cys and His residues in this domain still bound to UB chains with affinity nearly equivalent to that of the wild-type protein (FIGS. 8A, 8B; C179S, C182S, H195R, and H201R). However, a double mutant C179S/H195R exhibited completely abolished (K48-linked) or severely affected (K63-linked) binding. These results support a critical role for the OTU2 C-terminus in UB chain association. The predicted zinc finger motif likely does not exist because any of single mutations of the predicted residues involving zinc chelating would have disrupted the zinc finger and the C-terminal structure. As the OTU2 N-terminal part containing the OTU domain is critical for chain binding, OTU2 was treated with NEM to determine if the catalytic cysteine is essential. Association with both UB chain types was abolished when OTU2 was pre-incubated with NEM, suggesting a critical role of the catalytic cysteine in chain binding (FIG. 8B; OTU2-NEM). However, the catalytic cysteine (Cys16) is actually not essential for UB chain binding as the site-specific mutant bound UB chains as well as the wild-type protein (FIG. 8B; C16S). The deleterious effect of NEM on OTU2 UB chain binding indicates other Cys residues are likely critical. We further examined the role of two other available Cys residues (Cys63 and Cys99) in the N-terminal half of OTU2 in ubiquitin chain association. The association with both chain types was nearly completely abolished in OTU2 variants with single and double substitutions of these Cys residues (FIGS. 8A, 8B; C63S, C99S, and C63S/99S). While most known ubiquitin-binding domains and motifs, such as UIM and UBA, are formed from small and restricted regions of ubiquitin binding proteins containing these domains, thus the involvement of scattered critical residues/domains in UB chain association by OTU2 is unique and suggests that a novel structural domain formed from a broader sequence region is likely involved in UB-binding.

Although OTLD1a contains a UBA-like domain at C-terminus, a C-terminal truncated variant in which the UBA-like domain was removed exhibited K48-linked chain binding activity equivalent to that of the wild-type protein (FIGS. 9A, 9B; CM). Based on additional experiments with various terminal deletion variants, the ubiquitin chain binding region is likely positioned between residues 211-410; N-terminal deletion variants truncated before residue 211 and C-terminal deletion variants truncated after 410 bound K48-linked chains (FIGS. 9A, 9B; NΔ1-2 and CΔ1-4). An internal fragment (211-444) covering OTU (OTUf) also bound K48-linked chains.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. An isolated polypeptide capable of cleaving a peptide linkage located between the C-terminus of the distal ubiquitin (UB) and any of the first methionine (M1), the 48^{th} (K48) or the 63^{rd} lysine (K63) of the proximal UB in a UB chain conjugated with a substrate protein, comprising:
an amino acid sequence at least 85% identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

2. The isolated polypeptide of claim 1, wherein the polypeptide is isolated from Arabidopsis thaliana (A. thaliana).

3. The isolated polypeptide of claim 2, wherein the isolated polypeptide has the amino acid sequence at least 85% identical to SEQ ID NO: 1, and mutations at amino acid residues 92, 89 and 288 of the isolated polypeptide abolish its activity to cleave the peptide linkage located between the C-terminus of the distal UB and the M1, K48, or K63 of the proximal UB in the UB chain.

4. The isolated polypeptide of claim 2, wherein the isolated polypeptide has the amino acid sequence at least 85% identical to SEQ ID NO: 4, and a mutation at amino acid residue 136 of the isolated polypeptide abolish its activity to cleave the peptide linkage located between the C-terminus of the distal UB and the K48 or K63 of proximal UB in the UB chain.

5. The isolated polypeptide of claim 4, wherein the isolated polypeptide has the amino acid sequence of SEQ ID NO: 5.

6. The isolated polypeptide of claim 2, wherein the isolated polypeptide has the amino acid sequence at least 85% identical to SEQ ID NO: 6, and a mutation at amino acid residue 48 of the isolated polypeptide abolish its activity to cleave the peptide linkage located between the C-terminus of the distal UB and the K48 or K63 of proximal UB in the UB chain.

7. The isolated polypeptide of claim 2, wherein the isolated polypeptide has the amino acid sequence at least 85% identical to SEQ ID NO: 8 or 9, and cleaves the peptide linkage located between the C-terminus of the distal UB and the K63 of proximal UB in the UB chain, at about pH 9 to 9.5.

8. The isolated polypeptide of claim 2, wherein the isolated polypeptide has the amino acid sequence at least 85% identical to any of SEQ ID NO: 1, SEQ ID NO: 3, SEQ ID NO: 4, SEQ ID NO: 6, SEQ ID NO: 8, or SEQ ID NO: 9; and is capable of cleaving the peptide linkage located between the C-terminus of the distal UB and the K48 or 63 of proximal ubiquitin in the UB chain at about pH 7 to 8.

9. The isolated polypeptide of claim 1, wherein the isolated polypeptide has the amino acid sequence at least 85% identical to SEQ ID NO: 2, and two mutations at amino acid residues 63 and 99, or 179 and 195 of the isolated polypeptide abolish its activity to bind ubiquitin.

10. A kit for cleaving a peptide linkage located between the C-terminus of the distal ubiquitin (UB) and any of the first methionine (M1), the 48^{th} (K48) or the 63^{rd} lysine (K63) of the proximal UB in a UB chain conjugated with a substrate protein, comprising:
a container;
at least one of the isolated polypeptide of claim 1; and
a legend associated with the container and indicating how to use the at least one isolated polypeptide of claim 1 to direct the cleavage of the peptide linkage.

11. A method of deubiquitylation of a substrate protein having a conjugated UB chain, comprising:
subjecting the substrate protein having the conjugated UB chain to the action of an isolated polypeptide having an amino acid sequence at least 85% identical to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9 for a sufficient period of time, so as to allow the cleavage of the peptide linkage located between the C-terminus of the distal ubiquitin (UB) and any of the first methionine (M1), the 48^{th} (K48) or the 63^{rd} lysine (K63) of the proximal UB in the conjugated UB chain.

12. The method of claim 11, wherein the polypeptide is isolated from Arabidopsis thaliana (A. thaliana).

13. The method of claim 12, wherein the isolated polypeptide has the amino acid sequence at least 85% identical to SEQ ID NO: 1, and mutations at amino acid residues 92, 89 and 288 of the isolated polypeptide abolish its activity to cleave the peptide linkage located between the C-terminus of the distal UB and the M1, K48, or K63 of the proximal UB in the conjugated UB chain.

14. The method of claim 12, wherein the isolated polypeptide has the amino acid sequence at least 85% identical to SEQ ID NO:, and a mutation at amino acid residue 136 of the isolated polypeptide abolish its activity to cleave the peptide linkage located between the C-terminus of the distal UB and the K48 or K63 of the proximal UB in the conjugated UB chain.

15. The method of claim 14, wherein the isolated polypeptide has the amino acid sequence of SEQ ID NO: 5.

16. The method of claim 12, wherein the isolated polypeptide has the amino acid sequence at least 85% identical to SEQ ID NO: 6, and a mutation at amino acid residue 48 of the isolated polypeptide abolish its activity to cleave the peptide linkage located between the C-terminus of the distal UB and the K48 or K63 of the proximal UB in the conjugated UB chain.

17. The method of claim 12, wherein the isolated polypeptide has the amino acid sequence at least 85% identical to SEQ ID NO: 8 or 9, and cleaves the peptide linkage located between the C-terminus of the distal UB and the K63 of the proximal UB in the conjugated UB chain at about pH 9 to 9.5.

18. The method of claim 12, wherein the isolated polypeptide has the amino acid sequence at least 85% identical to any of SEQ ID NO: 1, SEQ ID NO: 3, SEQ ID NO: 4, SEQ ID NO: 6, SEQ ID NO: 8, or SEQ ID NO: 9; and is capable of cleaving the peptide linkage located between the C-terminus of the distal UB and the K48 or 63 of proximal ubiquitin in the conjugated UB chain at about pH 7 to 8.

19. The method of claim 12, wherein the isolated polypeptide has the amino acid sequence at least 85% identical to SEQ ID NO: 2, and two mutations at amino acid residues 63 and 99, or 179 and 195 of the isolated polypeptide abolish its activity to bind ubiquitin.


SEQUENCE LISTING (78 sequences)

SEQ ID NO: 1 - protein, 306 aa, Artificial Sequence
  OTU1-polypeptide
MQNQIDMVKDEAEVAASISAIKGEEWGNCSSVEDQPSFQEEEAAKVPYVGDKEPLSSLAAEYQSGSPILLEKIKILDSQYIGIRRTRGDGNCFFRSFMFSYLEHILESQDRAEVDRIKVNVEKCRKTLQNLGYTDFTFEDFFALFLEQLDDILQGTEESISYDELVNRSRDQSVSDYIVMFFRFVTAGDIRTRADFFEPFITGLSNATVDQFCKSSVEPMGEESDHIHITALSDALGVAIRVVYLDRSSCDSGGVTVNHHDFVPVGITNEKDEEASAPFITLLYRPGHYDILYPKPSCKVSDNVGK

SEQ ID NO: 2 - protein, 208 aa, Artificial Sequence
  OTU2-polypeptide
MEGIIVRRVIPSDNSCLFNAIGYVMDKDKNKAPELRQVIAAAVASNKEKYNEAFLGKLNEEYCAWILNPDKWGGAIELSILADYYGREIAAYDIQTSRCDLYGQTRNYDERVMLIYDGLHYDALALSPFEGAEEDFDMTIYPVGKDRSIGSIEGLALNLVKDQQRKRSYTDTANFTLRCGVCQIGVIGQKEAVEHAQATGHVNFQEYK

SEQ ID NO: 3 - protein, 234 aa, Artificial Sequence
  OTU3-polypeptide
MELKSSSNNNILEQLRNGFARFELVSSPTASVSDSISSTSLPASFISTTKGNSYVFFARINSSMNRSPAAKKVEKYAVDRVKGDGRCLFRALVKGMAFNKGITLNPQRERDDADELRMAVKEVICNDPKEREKYKEALVAITVDESLKRFCQRIGRHDFWGGESELLVLSKLCKQPIIVYIPEHEHGRGGGYGPGFIPIQEYGSEFRGGWGKGKTNKNVVRLLYSGRNHYDLLR

SEQ ID NO: 4 - protein, 274 aa, Artificial Sequence
  OTU4c-polypeptide
MMICYSPITTCSRNAISIKRHLGSRLYGVVAHGSSKFSCYSLLSGLSRRHYTGFRVSVSNRPSSWHDKGLFGFAYTGVIVSLLVCYSSTSQSAYADSSRDKDANNVHHHSSDGKFHNGKRVYTDYSIIGIPGDGRCLFRSVAHGFCLRSGKLAPGEKMQRELADELRTRVADEFIQRRQETEWFVEGDFDTYVRQIRDPHVWGGEPELFMASHVLQMPITVYMKDDKAGGLISIAEYGQEYGKDDPIRVLYHGFGHYDALLLHESKASIPKSKL

SEQ ID NO: 5 - protein, 231 aa, Artificial Sequence
  OTU4c-N?-polypeptide
SGLSRRHYTGFRVSVSNRPSSWHDKGLFGFAYTGVIVSLLVCYSSTSQSAYADSSRDKDANNVHHHSSDGKFHNGKRVYTDYSIIGIPGDGRCLFRSVAHGFCLRSGKLAPGEKMQRELADELRTRVADEFIQRRQETEWFVEGDFDTYVRQIRDPHVWGGEPELFMASHVLQMPITVYMKDDKAGGLISIAEYGQEYGKDDPIRVLYHGFGHYDALLLHESKASIPKSKL

SEQ ID NO: 6 - protein, 375 aa, Artificial Sequence
  OTU7a-polypeptide
MAKTKQQKSKPKKQPHQKQGKDCDLSQFRAQLDALGLKIIQVTADGNCFFRAIADQLEGNEDEHNKYRNMIVLYIVKNREMFEPFIEDDVPFEDYCKTMDDDGTWAGNMELQAASLVTRSNICIHRNMSPRWYIRNFEDTRTRMIHLSYHDGEHYNSVRSKEDACGGPARPVVIEADAKVSAASKQAKATESKSKNKADKCHVNAGAIKVVMSGSCCDNTEKAEQVLLQVNGDVDAAIEFLIADQGMESLTENDTETASASDTINPKHASDSPMENTEQAREELIEEESASGNNSETVQAKCTTQTDDKKIPRNKTCPCGSKKKYKSCCGTATGRSSVKLLVSQTMESKKGRKNLRRGTSNEVEANAPDVGALCI

SEQ ID NO: 7 - protein, 231 aa, Artificial Sequence
  OTU7b-polypeptide
MAKTKQQKSKPKKQPHQQGKDCDLSQFRAQLDALGLKIIQVTADGNCFFRAIADQLEGNEDEHNKYRNMIVLYIVKNREMFEPFIEDDVPFEDYCKTMDDDGTWAGNMELQAASLVTRSNICIHRNMSPRWYIRNFEDTRTRMIHLSYHDGEHYNSVRSKEDACGGPARPVVIEVLLQTLALCLSVIYINCLFFCPNKFQLCGRLMLKFQQHLNKRKLQRASPKIKLISVM

SEQ ID NO: 8 - protein, 345 aa, Artificial Sequence
  OTU9-polypeptide
MGYEPDPDALRWGLHDLEVCTLTNAGSCSSVTRYESGGGGTQGYVREGYNQPVTGYVDNDAVIAQFYQDELSRVARAEASGINSLSPTSVVAQDWPHPHQGQENQGEAIDITQESDILHNHNGNMEDKNVARIRFEGGQSSPSRDDDSVCSVEIEEESWSEVGKRLNQMIPIAHVPKINGELPSEDEQISDHERLFQRLQLYGLVENKIEGDGNCQFRSLSDQLYRSPEHHNFVREQVVNQLAYNREIYEGYVPMAYNDYLKAMKRNGEWGDHVTLQAAADLFGVRMFVITSFKDTCYIEILPHFQKSNRLICLSFWAEVHYNSIYPEGELPIPEGKKKKKYWVF

SEQ ID NO: 9 - protein, 356 aa, Artificial Sequence
  OTU10-polypeptide
MVSHEENTSIVEWFLGPHPYTYPPYGIEMIHEDDEVAVAHHHHHHQSGEYYREYEDHRSSDVDNDEIIARTLQDDFLQLEIAESNDYSHQNQQQQHQQEGYTNNYSNNNNGYAWNDQSPAVDYSSEWIGNDNDQDGRSDDSVNVFSCSSPSDTDEYVYSWESDQCDADGEFGRRLNQMVPIPYIPKINGEIPPEEEAVSDHERLRNRLEMFDFTEVKVPGDGNCQFRALADQLYKTADRHKHVRRQIVKQLKSRPDSYQGYVPMDFSDYLRKMSRSGEWGDHVTLQAAADAYRVKIVVLTSFKDTCYIEILPTSQESKGVIFLSFWAEVHYNAIYLNRDTSETELQRKRKWWRFGN

SEQ ID NO: 10 - DNA, 924 bp, Artificial Sequence
  OTU1-nucleic acid
atgcagaatcagattgatatggtgaaggatgaagcggaagtagctgcatcgatttcagcaattaagggtgaagaatggggaaattgttcatcagtggaagatcaaccatcttttcaagaagaagaagctgctaaagttccttatgttggtgataaggaacctctgtctagtttagctgcagagtatcaatcagggagtcccattttgctggagaagattaagatactggacagtcaatatatcggaatccggcgaacaagaggagatggaaattgcttcttccgaagttttatgttctcttaccttgagcatatattggaatcacaagatcgtgctgaagtcgatcgtatcaaggtcaatgttgagaaatgtagaaagactctgcaaaacttaggttatacagattttacatttgaggacttctttgcgttgttccttgagcaactagatgacattctccaaggaactgaagagtctataagctacgatgagctggttaacagaagtagagatcagtcagtctcagattacattgtaatgttctttaggtttgttactgctggtgatatacgaacgcgtgccgattttttcgagccttttataacaggcttatcaaatgcaacagtggatcagttttgcaagtcctcggtcgaaccaatgggggaagagagtgaccatattcacataactgctttgtcggacgcacttggtgttgcaatccgtgttgtgtatcttgaccgtagctcatgtgatagtgggggcgtcactgtgaatcatcatgactttgttcctgtgggcattaccaatgagaaagatgaagaagcttctgctccatttataaccttgctgtatcgtccaggccattacgatatcctctaccccaagccatcttgtaaggtatcagacaatgtggggaaatgatca

SEQ ID NO: 11 - DNA, 684 bp, Artificial Sequence
  OTU2-nucleic acid
atggaaggaatcattgtgagaagagttataccatctgataacagttgtctcttcaatgcaatcggttatgtcatggacaaggacaaaaacaaagctcctgagctcagacaggtgatagcagcagcagttgcaagcaacaaggagaaatataatgaagcatttctagggaagctcaatgaagaatattgtgcttggattctcaatccagacaagtggggaggtgcgattgagctttcgatattagcagattattatggtcgagaaattgcagcttacgacattcaaactagtcgatgtgacttgtatggacagacgagaaactacgacgaaagagttatgctgatctatgacggtcttcattacgatgctcttgctctgtctccatttgaaggggccgaggaagattttgatatgactatatatccagttggtaaagatagatccataggatcaattgaagggcttgctttgaatctagtgaaggaccaacaaaggaaaaggagttacacagatactgcaaacttcactctacgttgcggtgtttgccaaattggagttattggacaaaaggaagctgtggaacatgctcaagcaactggtcatgttaattttcaagaatacaaataagagaatgattgatttgattcttttatacagttccaagaaacaagaaagtaaaccacg

SEQ ID NO: 12 - DNA, 750 bp, Artificial Sequence
  OTU3-nucleic acid
atggagctcaaatcttcttccaacaataacattctagagcagctgagaaatggcttcgctagattcgagcttgtttcttctcctactgcttctgtgtcagattcaatatcttccacttcactccctgcttcattcataagcacaacgaagggaaatagttatgtgttcttcgccagaatcaattcctcgatgaatcggtctcctgcagcgaaaaaggttgagaagtatgctgtggatagagttaaaggggatgggagatgtctgtttcgagcattggtgaaaggaatggcctttaataagggtattactcttaacccccaacgagagagagatgatgcagatgagttacgaatggctgtgaaagaggttatatgcaatgaccccaaggaaagggagaagtacaaagaagctttagtggctatcactgtggatgagtctctgaaacgcttttgccagcggattggaagacacgatttctggggaggagagtccgagctgctagttctctccaagctatgtaaacagcctatcatcgtctacatacctgagcacgagcatggaagaggaggagggtatgggccgggttttataccgattcaagagtatggaagtgaatttcgaggaggctggggaaaggggaagacaaacaagaatgttgttaggcttctttacagtggtagaaaccactatgatttgcttcgttgaccaaattttttttttttgcctctgccgttgacatttattgatatg

SEQ ID NO: 13 - DNA, 870 bp, Artificial Sequence
  OTU4c-nucleic acid
atgatgatttgttactctccaattacaacatgttctaggaatgctatcagtatcaagaggcatttaggctctcgcctctacggcgttgtggctcacggatcatctaaattctcctgctacagtcttttatcaggactttcaagaagacattataccggttttcgtgtatcggtctctaatagaccttctagttggcacgataaaggactttttggatttgcatatacaggtgtgattgttagcttgcttgtttgttattcttcgacttcccaatcagcgtacgcagattcttcaagggataaagatgctaacaacgtgcatcatcattcgtctgatggtaaattccacaatgggaaaagagtttatactgattactctatcattggtattccgggggatggcagatgtttgtttcgttctgtagctcacggattctgcttacgctctgggaaactagctccaggcgagaagatgcagagagaactcgctgacgaattacgtaccagggttgctgatgagtttatccaaagaagacaggaaacagaatggttcgtggaaggagatttcgacacctacgtcagacaaattcgggatccacacgtgtggggaggtgaacctgaactcttcatggcttcacacgttctccagatgccaatcacagtgtatatgaaagacgataaagctggaggactgataagtattgcagaatatggtcaagaatacggtaaagacgacccaatccgagtcctataccatggttttggtcattacgatgcccttttgcttcacgagagtaaagcttcgattccaaaatctaaactttagtatacttagttttgcatctttgacttttgtcaggtgttgatgatg

SEQ ID NO: 14 - DNA, 741 bp, Artificial Sequence
  OTU4c-N?-nucleic acid
tcaggactttcaagaagacattataccggttttcgtgtatcggtctctaatagaccttctagttggcacgataaaggactttttggatttgcatatacaggtgtgattgttagcttgcttgtttgttattcttcgacttcccaatcagcgtacgcagattcttcaagggataaagatgctaacaacgtgcatcatcattcgtctgatggtaaattccacaatgggaaaagagtttatactgattactctatcattggtattccgggggatggcagatgtttgtttcgttctgtagctcacggattctgcttacgctctgggaaactagctccaggcgagaagatgcagagagaactcgctgacgaattacgtaccagggttgctgatgagtttatccaaagaagacaggaaacagaatggttcgtggaaggagatttcgacacctacgtcagacaaattcgggatccacacgtgtggggaggtgaacctgaactcttcatggcttcacacgttctccagatgccaatcacagtgtatatgaaagacgataaagctggaggactgataagtattgcagaatatggtcaagaatacggtaaagacgacccaatccgagtcctataccatggttttggtcattacgatgcccttttgcttcacgagagtaaagcttcgattccaaaatctaaactttagtatacttagttttgcatctttgacttttgtcaggtgttgatgatg

SEQ ID NO: 15 - DNA, 1160 bp, Artificial Sequence
  OTU7a-nucleic acid
atggcgaaaactaaacaacagaaatctaagcctaagaaacaaccgcatcagaagcaaggaaaggattgtgatctctctcaatttcgtgctcagcttgacgctttagggcttaagattatccaagtgactgctgacggtaattgtttcttcagagcaattgctgatcaattagaaggcaatgaggatgaacacaacaagtaccgtaacatgattgtactttatatagtgaaaaatcgtgaaatgttcgagccttttatcgaagatgatgttccttttgaggattactgtaaaaccatggacgatgatggaacttgggctgggaatatggaattacaagcagcttctcttgtcactcgcagtaatatatgtattcacaggaacatgtcaccgcgttggtatatacgcaactttgaagatacgcgaacccgcatgatccatctgtcttaccatgatggggaacactacaacagtgtgcgttccaaggaagatgcttgtggaggaccagccaggcctgttgtaatagaggctgatgctaaagtttcagcagcatctaaacaagcgaaagctacagagagcaagtccaaaaataaagctgataagtgtcatgtgaatgctggagctatcaaagttgtcatgtctggtagttgttgtgacaatactgagaaagctgaacaggttctattacaagttaatggtgatgtggatgctgctatagaattcctgatagcagaccaaggaatggagtctttgactgaaaatgatacagaaaccgcctctgcatcagataccatcaacccaaagcatgcaagtgatagtccaatggaaaacaccgagcaagccagagaagaattgatagaggaggaatcagctagcggcaataacagtgaaacagtccaagccaagtgcactacacagaccgatgataagaagatcccaaggaataaaacatgtccatgtggttcaaagaagaaatacaagtcctgctgtggaacagcaactgggagatcctctgtaaaactgttagtgagccaaacaatggagtcgaagaaggggagaaagaatttgagaagaggaacatcgaatgaagttgaagcaaatgcaccagatgttggtgctctttgtatataattaagtctcactcactcaaggcgaagacataa

SEQ ID NO: 16 - DNA, 1246 bp, Artificial Sequence
  OTU7b-nucleic acid
atggcgaaaactaaacaacagaaatctaagcctaagaaacaaccgcatcagcaaggaaaggattgtgatctctctcaatttcgtgctcagcttgacgctttagggcttaagattatccaagtgactgctgacggtaattgtttcttcagagcaattgctgatcaattagaaggcaatgaggatgaacacaacaagtaccgtaacatgattgtactttatatagtgaaaaatcgtgaaatgttcgagccttttatcgaagatgatgttccttttgaggattactgtaaaaccatggacgatgatggaacttgggctgggaatatggaattacaagcagcttctcttgtcactcgcagtaatatatgtattcacaggaacatgtcaccgcgttggtatatacgcaactttgaagatacgcgaacccgcatgatccatctgtcttaccatgatggggaacactacaacagtgtgcgttccaaggaagatgcttgtggaggaccagccaggcctgttgtaatagaggttttacttcaaactttagctctgtgtttatctgtaatatacataaattgtctgtttttctgtcctaataaattccagttgtgtggaaggctgatgctaaagtttcagcagcatctaaacaagcgaaagctacagagagcaagtccaaaaataaagctgataagtgtcatgtgaatgctggagctatcaaagttgtcatgtctggtagttgttgtgacaatactgagaaagctgaacaggttctattacaagttaatggtgatgtggatgctgctatagaattcctgatagcagaccaaggaatggagtctttgactgaaaatgatacagaaaccgcctctgcatcagataccatcaacccaaagcatgcaagtgatagtccaatggaaaacaccgagcaagccagagaagaattgatagaggaggaatcagctagcggcaataacagtgaaacagtccaagccaagtgcactacacagaccgatgataagaagatcccaaggaataaaacatgtccatgtggttcaaagaagaaatacaagtcctgctgtggaacagcaactgggagatcctctgtaaaactgttagtgagccaaacaatggagtcgaagaaggggagaaagaatttgagaagaggaacatcgaatgaagttgaagcaaatgcaccagatgttggtgctctttgtatataattaagtctcactcactcaaggcgaagacataa

SEQ ID NO: 17 - DNA, 1042 bp, Artificial Sequence
  OTU9-nucleic acid
atggggtatgagcctgatccagatgctcttaggtggggtcttcatgatctggaggtttgtacacttacaaatgctggttcttgctctagtgtaacaaggtatgagtctggtggtggtggtactcaaggctatgttagagaaggttacaatcagcctgtgactggttatgttgacaatgatgcggtgattgctcagttttatcaagacgaattgtctagagttgctcgagccgaagcatcggggatcaatagtcttagtcctacttctgttgttgcacaagactggcctcatcctcatcaaggacaggaaaatcaaggggaagccattgatattactcaggaaagtgatattcttcataatcataacggtaacatggaagataagaatgttgcaaggataagatttgaaggagggcaaagttccccatctcgtgatgatgactcagtttgttctgtagaaatagaagaagaatcttggtcagaggttgggaagcgactgaaccaaatgatacctattgctcatgtgccgaaaatcaatggggaattaccgtcagaggatgagcagatttctgatcatgaacggctatttcagagattacagctttatggcttggtggagaacaagattgaaggagatggaaactgccagtttcggtcactatcagaccagctttaccgctctccagaacatcataattttgtcagagaacaagttgttaatcagcttgcatataatcgagagatatatgaaggttatgttccaatggcgtacaatgactatcttaaagcaatgaaacggaatggagaatggggtgatcacgttactcttcaggctgctgcggatttgtttggtgttcggatgtttgtgataacttcattcaaggatacatgctacattgagatcttgcctcattttcagaagtctaatcgccttatatgtttaagcttctgggctgaggttcattacaattcaatctaccctgaaggagagctgccgataccagaaggcaagaagaagaagaaatattgggtattctgagctc

SEQ ID NO: 18 - DNA, 1079 bp, Artificial Sequence
  OTU10-nucleic acid
atggtgtcacatgaagagaacacaagtattgtggaatggtttcttggtccacatccatacacgtatccgccttatggtattgagatgattcatgaggatgatgaagtagcagtagctcatcatcatcatcatcaccagagcggtgagtattacagagagtatgaagatcatcgtagtagcgatgtagataacgatgagatcattgctagaactctccaagacgattttttgcagcttgaaatcgcagaaagtaacgattattcgcatcagaatcaacaacaacaacatcaacaagaaggttatactaacaactatagtaataacaacaatggttatgcttggaatgatcagtctcctgctgtggattattcttcagaatggataggaaatgataatgatcaagatgggaggagcgatgattcggttaatgtcttttcgtgttcaagcccgagtgacacggatgagtatgtatactcgtgggaatcggatcagtgtgatgctgatggtgaatttggaaggagattgaatcaaatggttcccattccgtatataccgaaaataaatggagaaatacctccggaagaagaagcagtttcagatcatgaaagacttcggaataggttagagatgtttgacttcactgaggttaaggttccaggagatggtaattgccagttccgtgctttagctgatcaactgtacaaaaccgcagatcgccataaacatgttcgacgacaaatagtaaaacagctaaaatctcgtccagattcttatcaaggatacgttcctatggacttctctgattatctaagaaagatgtctcggagcggcgaatggggcgatcatgtgacattacaggcagctgcagatgcgtatcgggtgaaaatagtagtcctaacatcgttcaaagatacatgttacattgaaattcttcctacctcccaagaatccaaaggagtaatcttcttgagcttctgggcagaggttcattacaacgccatctacttaaacagagatacatctgaaacagagctacagaggaagagaaaatggtggcgtttcggaaactagacagacca

SEQ ID NO: 19 - DNA, 32 bp, Artificial Sequence
  OTU1-F primer
taggatccatgcagaatcagattgatatggtg

SEQ ID NO: 20 - DNA, 37 bp, Artificial Sequence
  OTU1-R primer
tatctcgagtgatcatttccccacattgtctgatacc

SEQ ID NO: 21 - DNA, 33 bp, Artificial Sequence
  OTU1-D89E-F primer
ggcgaacaagaggagaaggaaattgcttcttcc

SEQ ID NO: 22 - DNA, 33 bp, Artificial Sequence
  OTU1-D89E-R primer
ggaagaagcaatttccttctcctcttgttcgcc

SEQ ID NO: 23 - DNA, 38 bp, Artificial Sequence
  OTU1-C92S-F primer
caagaggagatggaaatagcttcttccgaagttttatg

SEQ ID NO: 24 - DNA, 38 bp, Artificial Sequence
  OTU1-C92S-R primer
cataaaacttcggaagaagctatttccatctcctcttg

SEQ ID NO: 25 - DNA, 33 bp, Artificial Sequence
  OTU1-H288R-F primer
ctgtatcgtccaggccgttacgatatcctctac

SEQ ID NO: 26 - DNA, 33 bp, Artificial Sequence
  OTU1-H288R-R primer
gtagaggatatcgtaacggcctggacgatacag

SEQ ID NO: 27 - DNA, 34 bp, Artificial Sequence
  OTU2-F primer
tatggatccatggaaggaatcattgtgagaagag

SEQ ID NO: 28 - DNA, 39 bp, Artificial Sequence
  OTU2-R primer
catctcgagcgtggtttactttcttgtttcttggaactg

SEQ ID NO: 29 - DNA, 35 bp, Artificial Sequence
  OTU3-F primer
tccggatccatggagctcaaatcttcttccaacaa

SEQ ID NO: 30 - DNA, 36 bp, Artificial Sequence
  OTU3-R primer
ttcctcgagcatatcaataaatgtcaacggcagagg

SEQ ID NO: 31 - DNA, 35 bp, Artificial Sequence
  OTU4a-F primer
atagaattcatgatgatttgttactctccaattac

SEQ ID NO: 32 - DNA, 35 bp, Artificial Sequence
  OTU4a-R primer
aaactcgagcatcatcaacacctgacaaaagtcaa

SEQ ID NO: 33 - DNA, 35 bp, Artificial Sequence; identical to SEQ ID NO: 31
  OTU4c-F primer

SEQ ID NO: 34 - DNA, 35 bp, Artificial Sequence; identical to SEQ ID NO: 32
  OTU4c-R primer

SEQ ID NO: 35 - DNA, 35 bp, Artificial Sequence; identical to SEQ ID NO: 31
  OTU4d-F primer

SEQ ID NO: 36 - DNA, 35 bp, Artificial Sequence; identical to SEQ ID NO: 32
  OTU4d-R primer

SEQ ID NO: 37 - DNA, 34 bp, Artificial Sequence
  GST-OTU4c-C136S-F primer
ccgggggatggcagaagtttgtttcgttctgtag

SEQ ID NO: 38 - DNA, 34 bp, Artificial Sequence
  GST-OTU4c-C136S-R primer
ctacagaacgaaacaaacttctgccatcccccgg

SEQ ID NO: 39 - DNA, 35 bp, Artificial Sequence
  OTU5a-F primer
cttgaattcatggcagacaataaaacatctgaagg

SEQ ID NO: 40 - DNA, 36 bp, Artificial Sequence
  OTU5a-R primer
gatgagctccatcatccatcatccggtgatgttgtt

SEQ ID NO: 41 - DNA, 38 bp, Artificial Sequence
  OTU5b-F primer
gaagaattccccatggatgcttctcaggagcaacaaca

SEQ ID NO: 42 - DNA, 36 bp, Artificial Sequence; identical to SEQ ID NO: 40
  OTU5b-R primer

SEQ ID NO: 43 - DNA, 35 bp, Artificial Sequence
  GST-OTLD1Aa-F primer
agagaattcatgactcggattttggttcaaagagg

SEQ ID NO: 44 - DNA, 33 bp, Artificial Sequence
  GST-OTLD1Aa-R primer
aatctcgagcaattcatgttacattctctattc

SEQ ID NO: 45 - DNA, 39 bp, Artificial Sequence
  OTU7a-F primer
gatggatccatggcgaaaactaaacaacagaaatctagg

SEQ ID NO: 46 - DNA, 37 bp, Artificial Sequence
  OTU7a-R primer
atactcgagttatgtcttcgccttgagtgagtgagac

SEQ ID NO: 47 - DNA, 40 bp, Artificial Sequence
  OTU7a-C48S-F primer
gtgactgctgacggtaatagtttcttcagagcaattgctg

SEQ ID NO: 48 - DNA, 40 bp, Artificial Sequence
  OTU7a-C48S-R primer
cagcaattgctctgaagaaactattaccgtcagcagtcac

SEQ ID NO: 49 - DNA, 34 bp, Artificial Sequence
  OTU9-F primer
gaattcatggggtatgagcctgatccagatgctc

SEQ ID NO: 50 - DNA, 26 bp, Artificial Sequence
  OTU9-R primer
cctccatcagcagaaagagagctcag

SEQ ID NO: 51 - DNA, 35 bp, Artificial Sequence
  OTU10-F primer
tgtgaattcatggtgtcacatgaagagaacacaag

SEQ ID NO: 52 - DNA, 37 bp, Artificial Sequence
  OTU10-R primer
atgaagctttggtctgtctagtttccgaaacgccacc

SEQ ID NO: 53 - DNA, 37 bp, Artificial Sequence
  OTU11a-F primer
ccaggatccatggatgaaaaccataggaatccatttg

SEQ ID NO: 54 - DNA, 35 bp, Artificial Sequence
  OTU11a-R primer
actgtcgacccaatactagaagagccaatgcttcc

SEQ ID NO: 55 - DNA, 35 bp, Artificial Sequence
  OTU12-F primer
cttgaattcatgggagactcttcgagttcaacctc

SEQ ID NO: 56 - DNA, 36 bp, Artificial Sequence
  OTU12-R primer
gcgaagcttctagaacaaccaatgttttctctttgg

SEQ ID NO: 57 - DNA, 33 bp, Artificial Sequence
  HsOTUB1-F primer
tgtggatccatggcggcggaggaacctcagcag

SEQ ID NO: 58 - DNA, 33 bp, Artificial Sequence
  HsOTUB1-R primer
gggctcgagccagccctatttgtagaggatatc

SEQ ID NO: 59 - DNA, 36 bp, Artificial Sequence
  UB2-F primer
gtgggatccatgcagatctttgttaagactctcacc

SEQ ID NO: 60 - DNA, 36 bp, Artificial Sequence
  UB2 R primer
gataagcttgaagaagttcgacttgtcattagaaag

SEQ ID NO: 61 - DNA, 36 bp, Artificial Sequence; identical to SEQ ID NO: 59
  UB3-F primer

SEQ ID NO: 62 - DNA, 36 bp, Artificial Sequence; identical to SEQ ID NO: 60
  UB3-R primer

SEQ ID NO: 63 - DNA, 36 bp, Artificial Sequence; identical to SEQ ID NO: 59
  UB4-F primer

SEQ ID NO: 64 - DNA, 36 bp, Artificial Sequence; identical to SEQ ID NO: 60
  UB4-R primer

SEQ ID NO: 65 - DNA, 35 bp, Artificial Sequence
  RAD23d-1-F primer
cccggatccatgaagattttcgtgaagactctcag

SEQ ID NO: 66 - DNA, 36 bp, Artificial Sequence
  RAD23d-1-R primer
ttgctcgagttattgatcttcaaactcatgcatgtg

SEQ ID NO: 67 - DNA, 35 bp, Artificial Sequence
  RAD23d-2-F primer
cgcaagcttatgaagattttcgtgaagactctcag

SEQ ID NO: 68 - DNA, 36 bp, Artificial Sequence
  RAD23d-2-R primer
gtgctcgagttattgatcctcaaactcatgcatgtg

SEQ ID NO: 69 - DNA, 32 bp, Artificial Sequence
  GST-F primer
gataagcttatgtcccctatactaggttattg

SEQ ID NO: 70 - DNA, 35 bp, Artificial Sequence
  GST-R primer
aacctcgagttcaaccatccgattttggaggatgg

SEQ ID NO: 71 - DNA, 30 bp, Artificial Sequence
  HA3-UBQ-F primer
ggacatatgcagatctttgttaagactctc

SEQ ID NO: 72 - DNA, 33 bp, Artificial Sequence
  HA3-UBQ-R primer
gaaaagcttgaaacctccacgcagacgcaacac

SEQ ID NO: 73 - DNA, 35 bp, Artificial Sequence
  RUB-F primer
ggtggatccatgattaaggtgaagactctcacagg

SEQ ID NO: 74 - DNA, 42 bp, Artificial Sequence
  RUB-R primer
tcaggcggccgctcaaccacccctaagggcaagaaccagatg

SEQ ID NO: 75 - DNA, 32 bp, Artificial Sequence
  HA3-RUB-1-F primer
gggcatatgattaaggtgaagactctcacagg

SEQ ID NO: 76 - DNA, 48 bp, Artificial Sequence
  HA3-RUB-1-R primer
tacaagcttgagaagaccaaaaccacccctaagggcaagaaccagatg

SEQ ID NO: 77 - DNA, 33 bp, Artificial Sequence
  HA3-SUMO-1-F primer
ttccatatgtctgcaaaccaggaggaagacaag

SEQ ID NO: 78 - DNA, 36 bp, Artificial Sequence
  HA3-SUMO-1-R primer
gaaaagcttggccgtagcaccaccaccgctgccacc